(19) United States
(12) Patent Application Publication
(10) Pub. No.: US 2011/0293526 A1
(43) Pub. Date: Dec. 1, 2011

(54) COMPOSITIONS AND METHODS TO MODULATE HAIR GROWTH

(75) Inventors: Maksim Plikus, Los Angeles, CA (US); Cheng Ming Chuong, Los Angeles, CA (US)

(73) Assignee: University of Southern California

(21) Appl. No.: 13/130,288

(22) PCT Filed: Nov. 19, 2009
(86) PCT No.: PCT/US09/65201

§ 371 Date: Aug. 8, 2011

Related U.S. Application Data
(60) Provisional application No. 61/116,619, filed on Nov. 20, 2008.

(51) Int. Cl. A61K 49/00; A61K 31/713; A61K 39/395; A61K 38/18; C12N 5/071; C12Q 1/68; C40B 30/04; A61K 31/519; A61P 17/00; A61P 17/14; A61K 38/17; C12Q 1/02

(52) U.S. Cl. 424/9.2; 514/8.8; 514/44 A; 424/130.1; 435/29; 435/6.12; 424/158.1; 506/9; 514/259.3; 435/375

(57) ABSTRACT

The invention provides compositions and methods to modulate hair growth in a tissue comprising administering to the tissue an effective amount of an agent that inhibits or augments Bone Morphogenic Protein (BMP) signaling in the tissue, thereby facilitating or inhibiting hair growth.

### CROSS-REFERENCE TO RELATED APPLICATIONS

This application claims the benefit under 35 U.S.C. §119(e) of U.S. Provisional Application Ser. No. 61/116,619, filed Nov. 20, 2008, the content of which is incorporated by reference into the present disclosure in its entirety.

### STATEMENT OF FEDERAL SUPPORT

This invention was supported by grants from the National Institutes of Health (Grant Nos. AR 42177 and AR 47354). The government has rights in this application.

### BACKGROUND

The skin is the second largest organ in the body. The skin of a mammal is derived from ectoderm and mesoderm layers of an embryo. These two layers give rise to the epidermis and dermis, respectively. The ectoderm and mesoderm layers also give rise to specialized appendages including sensory nerves, sweat glands, and hair follicles.

Excessive hair (hirsutism) and hair loss (alopecia) are two conditions associated with the skin. Hirsutism is defined as excessive and increased hair growth in locations where the occurrence of terminal hair normally is minimal or absent. It is primarily of cosmetic and psychological concern. The most common form of hair loss (aka alopecia) in men is male pattern baldness (aka androgenic alopecia). In the case of androgenic alopecia, hair loss occurs gradually over several years. It usually starts on the crown of the head and progresses toward the forehead area. In women suffering from alopecia, hair loss occurs in a more dispersed pattern with thinning of the scalp hair and commonly appears following the menopause. Studies to develop a substance for alleviating or treating alopecias of different etiology, particularly a substance for stimulating hair growth or reducing hair loss, have been made from long ago in the cosmetic or pharmaceutical industry field.

Many people with unwanted hair seek methods of hair removal to control the appearance of hirsutism. There are not many pharmaceutical treatments available for hirsutism, one of which is the antiandrogen drug Spironolactone. Side effects of Spironolactone include increased risk of bleeding from the stomach and duodenum, gynecomastia, menstrual irregularities, testicular atrophy, ataxia, erectile dysfunction, drowsiness and rashes. Laser hair removal or hair electrolysis can be used and these are generally effective. Laser hair removal, however, kills follicles rather than converts terminal follicle back into velus. Later conversion may be desirable in some cases.

For alopecia, a large number of compounds have been developed as candidate treatments. Examples include 2,4-diamino-6-piperidinopyrimidine-3-oxide (also known as “minoxidil”) and finasteride as disclosed in U.S. Pat. No. 4,139,619 and U.S. Pat. No. 4,596,812, respectively. A medicament containing minoxidil as an active ingredient is commercially available under the trademark “Rogaine” (Pharmacia & Upjohn Company). A medicament containing finasteride as an active ingredient is commercially available under the trademark “Propecia” (Merck & Co., Inc.). Propecia is a pill for oral administration. Both treatments require continuous application of the compositions to the skin for a long period of time and the success rates are limited.

Attempts have been made to extracts compositions from natural plants, including medicinal herbs, to be used for the treatment of alopecia. Various extracts of crude drugs, generally known as hair growth compositions, have been used as hair growth stimulants or promoters. Even though some of these hair growth compositions show some effects, the treatments come with some common adverse effects such as skin irritation and unpleasant odor.

Another method for treatment of alopecia is hair transplantation. This method typically comprises transplanting the natural hair in the scalp area where hair grows to the bald area. Hair transplantation often times is costly, time consuming, painful and only limitedly successful.

There is a need for the development of a method and a composition to enhance or inhibit hair growth, therefore efficiently treating alopecia or hirsutism.

### SUMMARY OF THE INVENTION

This invention provides a method for facilitating hair growth in a tissue containing hair follicles comprising, or alternatively consisting essentially of, or yet further consisting of, administering to the tissue an effective amount of an agent that inhibits Bone Morphogenic Protein (BMP) signaling in the tissue, thereby facilitating hair growth. In one aspect, the agent is dorsomorphin and/or noggin. In another aspect, the agent is administered during the telogen stage of hair regeneration and growth. In a further aspect is it administered during telogen stage of hair regeneration and growth. In another aspect, the agent is administered in combination with agent that facilitates hair growth such as minoxidil, finasteride and/or spironolactone. The second agent can be co-administered or administered prior to or subsequent to administration of the agent that inhibits BMP signaling in the tissue. When co-application will be done with other agents, substance such as Minoxidil will be applied simultaneously. If another substance is already known to specifically boost (lengthen) growth phase and/or result in thicker hair formation (vellus-to-terminal hair transformation), such substance can be administered after BMP inhibitors. The agents can be administered topically, intradermally or ingested as appropriate.

In another aspect, this invention provides a method for treating alopecia in a subject having tissue containing a hair follicle, comprising, or alternatively consisting essentially of, or yet further consisting of, administering to the tissue an effective amount of an agent that inhibits Bone Morphogenic Protein (BMP) signaling to the tissue, thereby treating alopecia in the subject. In one aspect, the agent is the agent is dorsomorphin and/or noggin. In another aspect, the agent is administered during the telogen stage of hair regeneration and growth. In a further aspect it is administered during the competent telogen stage of hair regeneration and growth. In another aspect, the agent is administered in combination with agent that facilitates hair growth such as minoxidil, finasteride and/or spironolactone. The second agent can be co-administered or administered prior to or subsequent to administration of the agent that inhibits BMP signaling in the tissue. When co-application will be done with other agents, substance such as Minoxidil will be applied simultaneously. If another substance is already known to specifically boost (lengthen) growth phase and/or result in thicker hair formation (vellus-to-terminal hair transformation), such substance can be administered after BMP inhibitors. The agents can be administered topically, intradermally or ingested as appropriate.

In another aspect, this invention provides a method for inhibiting hair growth in a tissue containing a hair follicle comprising, or alternatively consisting essentially of, or yet further consisting of, administering to the tissue an effective amount of an agent that augments Bone Morphogenic Protein (BMP) in the tissue, thereby inhibiting hair growth. In another aspect, the agent is administered in combination with agent that suppresses hair growth. Agonists include repulsive guidance molecule (RGMA), DRAGON (RGMB), hemojuvelin, kielin/chordin-like protein (KCP), and Crossveinless 2 (Cv2). The second agent can be co-administered or administered prior to or subsequent to administration of the agent that inhibits BMP signaling in the tissue. The agents can be administered topically, intradermally or ingested as appropriate.

In another aspect, this invention provides a method for treating hirsutism in a subject having tissue containing a hair follicle, comprising, or alternatively consisting essentially of, or yet further consisting of, administering to the tissue an effective amount of an agent that augments Bone Morphogenic Protein (BMP) in the tissue, thereby treating hirsutism in the subject. In another aspect, the agent is administered in combination with agent that suppresses hair growth. Agonists include repulsive guidance molecule (RGMA), DRAGON (RGMB), hemojuvelin, kielin/chordin-like protein (KCP), and Crossveinless 2 (Cv2). The agents can be administered topically, intradermally or ingested as appropriate.

In yet another aspect, this invention provides a composition comprising, or alternatively consisting essentially of, or yet further consisting of, an effective amount of an agent that augments BMP signaling and a pharmaceutically acceptable carrier. The composition can further comprise, or alternatively consist essentially of, or yet further consist of, an effective amount of a second agent such as spironolactone. The compositions can be formulated for topical or interdermal administration, or ingested as appropriate.

In yet another aspect, this invention provides a composition comprising, or alternatively consisting essentially of, or yet further consisting of, an effective amount of an agent that inhibits BMP signaling and a pharmaceutically acceptable carrier. The composition can further comprise, or alternatively consist essentially of, or yet further consist of, an effective amount of a second agent such as minoxidil, finasteride and/or spironolactone.

In yet another aspect, this invention provides a method to determine if a test agent will likely modulate hair growth in a tissue having a hair follicle, comprising, or alternatively consisting essentially of, or yet further consisting of, (a) administering to a first tissue sample an amount of the test agent; (b) administering to a second tissue sample an effective amount of soluble BMP and/or (c) administering to a third tissue sample an effective amount of BMP antagonist, such as noggin; and (d) comparing the growth of hair in the first tissue sample to the growth in the second tissue sample and/or third tissue sample, wherein the test agent will likely modulate hair growth if the growth of hair in the first tissue sample is similar to the second tissue sample and/or third sample. In another aspect, the agent is administered in combination with agent that modulates hair growth such as minoxidil, finasteride and/or spironolactone. The second agent can be co-administered or administered prior to or subsequent to administration of the agent that modulates BMP signaling in the tissue. Timing of the agent delivery can be further modified, e.g. by administering during the telogen phase of hair growth and/or regeneration.

Further provided is use of the above-mentioned compositions in the manufacture of a medicament for modulating hair growth. In one aspect, the medicament will facilitate hair growth. In another aspect, the medicament will inhibit or diminish hair growth. The medicaments may further comprise additional pharmaceuticals or agents that facilitate or alternatively, inhibit hair growth, e.g., minoxidil, finasteride or spironolactone. These may be combined with pharmaceutically acceptable carriers that are suitable for the modes of administration.

In yet another aspect, this invention provides a kit for inhibiting hair growth in a tissue having a hair follicle, comprising, or alternatively consisting essentially of, or yet further consisting of, an effective amount of an agent that augments BMP in a pharmaceutically acceptable carrier and instructions for use in inhibiting hair growth.

In yet another aspect, this invention provides a kit for augmenting or promoting hair growth comprising an effective amount of an agent that inhibits BMP in a pharmaceutically acceptable carrier and instructions for use in augmenting or promoting hair growth.

### BRIEF DESCRIPTION OF THE FIGURES

FIG. 1 is a schematic summary of the hair-cycle rhythm (dark) and the newly identified dermal rhythm (gray). Together, they define four new functional stages. Catagen is omitted for simplification.

FIG. 2 a, Control (left) and Krt14-Nog (right) mice. Hair-cycle domains in two different stages are shown, together with schematic domain boundaries. b, Measurements show that both refractory and competent telogen are shortened in Krt14-Nog mice (K14N, gray bars) compared to wild type (WT, dark bars). In b and d, Min and Max represent range of values, whereas numbers at the bottom represent average number of days. In c, however, numbers at the bottom represent numbers of plucked hairs. Error bars, standard deviation; n=71 for Nog mice and n=22-30 for the control. c, Plucking/regenerative response in Krt14-Nog (gray bars) is about 5 times faster. d, e, When a small Krt14-Nog skin graft was transplanted into SCID skin, hair growth (e) and duration of refractory telogen (d) were partially rescued (error bars, standard deviation; n>15). The gray dotted line represents the anagen wave front. Gray arrows point at the transplanted Krt14-Nog hair follicles. The blank arrow points at the spreading direction of the anagen wave. The blank arrowhead points at the enlarged view of the top panel. f, When a large Krt14-Nog skin graft (>10 mm) was transplanted, it caused reduction of refractory telogen by inducing a rim of white hair in the host. g, h, Human-BMP4-soaked beads caused hair propagation wave (solid arrow) to go around them, creating a new telogen domain. Albumin does not have this effect. Dashed line, domain border. Scale bars: e, g, h, 1 mm.

FIG. 3 shows interactions of small KRT14-NOG skin transplant with the host skin macro-environment. When a small graft of KRT14-NOG skin (˜1 mm in diameter) was transplanted, the donor skin remained in telogen longer (a) and could respond to an anagen activating wave originating from the host (b). Thus partial functional rescue of KRT14-NOG phenotypes was achieved. On some occasions, some grafts exhibited a greater degree of autonomous control (c) and can induce host hair follicles surrounding the perimeter of KRT14-NOG skin graft into anagen (d). Pigmented hairs are from donor KRT14-NOG. White hairs are from SCID mice.

FIG. 4 shows that BMP protein can convert competent telogen status to refractory. (a) hBMP4-soaked beads caused hair propagation wave (solid arrowed curve) to go around them, creating a new telogen domain. (b) Albumin does not have this effect. Broken line, domain border.

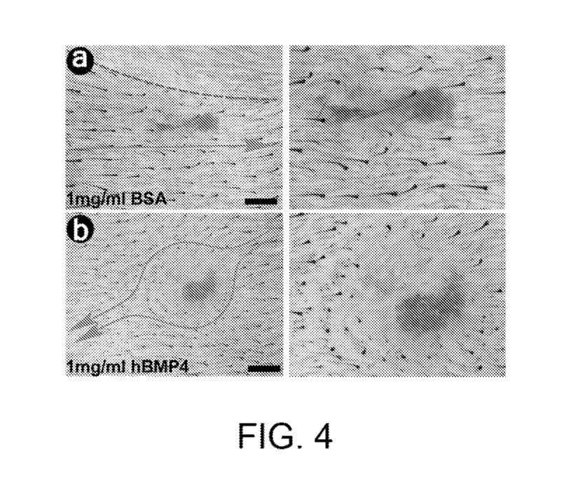

FIG. 5 a illustrates the bulge niche microenvironment and interfollicular dermal macroenvironment, including dermis, subcutaneous fat and adjacent follicles. Anagen-stimulating or -inhibiting activities are depicted with arrows with a flat bar at the end indicating inhibition and an arrowhead stimulation. Follicles are in different stages: A, refractory telogen; B, competent telogen; C, propagating anagen; and D, autonomous anagen follicles. The upper left circle in A indicates intrafollicular microenvironment. FIG. 5 b illustrates new functional phases (outer circle) mapped against classical hair-cycle stages (inner circle). On the basis of the growth-inducing ability of the follicles, anagen is divided into propagating (inducing, upper left portion of the outer circle) and autonomous (non-inducing, upper right of the outer circle) phases. On the basis of the ability to respond to regenerative signals, telogen is divided into refractory telogen (bottom right portion of the outer circle) and competent (bottom left portion of the outer circle) phases.

### DETAILED DESCRIPTION OF THE INVENTION

Throughout this disclosure, various publications, patents and published patent specifications are referenced by an identifying citation. The disclosures of these publications, patents and published patent specifications are hereby incorporated by reference in their entirety into the present disclosure to more fully describe the state of the art to which this invention pertains.

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; and Animal Cell Culture (R. I. Freshney, ed. (1987)).

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

“Bone Morphogenic Proteins” (BMP) are a group of multifunctional growth factors and cytokines with effects in various tissues. For example, BMPs are known to induce the formation of bone and/or cartilage. Examples of BMP may include, but are not limited to BMP1, BMP2, BMP3, BMP4, BMP5, BMP6, BMP7, BMP8a, BMP8b, BMP10 and BMP15.

“BMP signaling” or “BMP signaling pathway” refers to the enzyme linked receptor protein signaling transduction pathway involving proteins that directly or indirectly regulate (activate or inhibit) downstream protein activity or gene expression. Examples of molecules involved in the BMP signaling pathways may be found in the public Gene Ontology (GO) database, under GO ID: GO:0030509, accessible at the web page (amigo.geneontology.org/cgi-bin/amigo/term-details.cgi?term=GO:0030509&session_id=5573amigo1226631957), last accessed on Nov. 17, 2008. Without limitation, examples of proteins in the BMP signaling pathway include Activin receptor type-1 (ACVR1, UniProt: Q04771), Activin receptor type-2A (ACVR2A, UniProt: P27037), Activin receptor type-2B (ACVR2B, UniProt: Q13705), BMP1 (UniProt: P13497), BMP2 (UniProt: P12643), BMP3 (UniProt: P12645), BMP4 (UniProt: P12644), BMP5 (UniProt: P22003), BMP6 (UniProt: P22004), BMP7 (UniProt: P18075), BMP8a (UniProt: Q7Z5Y6), BMP8b (UniProt: P34820), BMP10 (UniProt: O95393), BMP15 (UniProt: O95972), Bone morphogenetic protein receptor type-1A (BMPR1A, UniProt: P36894), Bone morphogenetic protein receptor type-1B (BMPR1B, UniProt: O00238), Bone morphogenetic protein receptor type-2 (BMPR2, UniProt: Q13873), Chordin-like protein (CHRDL1, UniProt: Q9BU40), Follistatin-related protein 1 (FSTL1, UniProt: Q12841), Growth/differentiation factor 2 (GDF2, UniProt: Q9UK05), Growth/differentiation factor 6 (GDF6, UniProt: Q6KF10), Growth/differentiation factor 7 (GDF7, UniProt: Q7Z4P5), Gremlin-2 (GREM2, UniProt: Q9H772), RGM domain family member B (RGMB, UniProt: Q6NW40), Ski oncogene (SKI, UniProt: P12755), Mothers against decapentaplegic homolog 4 (SMAD4, UniProt: Q13485), Mothers against decapentaplegic homolog 5 (SMAD5, UniProt: Q99717), Mothers against decapentaplegic homolog 6 (SMAD6, UniProt: O43541), Mothers against decapentaplegic homolog 7 (SMAD7, UniProt: O15105), Mothers against decapentaplegic homolog 9 (SMAD9, UniProt: O15198), E3 ubiquitin-protein ligase SMRF2 (SMURF2, UniProt: Q9HAU4), TGF-beta receptor type III (TGFBR3, UniProt: Q03167), Ubiquitin-conjugating enzyme E2 D1 (UBE2D1, UniProt: P51668), Ubiquitin-conjugating enzyme E2 D3 (UBE2D3, UniProt: P61077) and Zinc finger FYVE domain-containing protein 16 (ZFYVE16, UniProt: Q7Z3T8). Proteins that positively or negatively regulate the BMP signaling, for purpose of this invention, are also considered within the meaning of the BMP signaling. Proteins that positively regulate BMP signaling include, but are not limited to, Serine/threonine-protein kinase receptor R3 (ACVRL1, UniProt: P37023) and Endoglin (ENG, UniProt: P17813). Proteins that negatively regulate BMP signaling include, but are not limited to, Chordin (CHRD, UniProt: Q9H2X0), E3 ubiquitin-protein ligase SMURF1 (SMURF1, UniProt: Q9HCE7), Sclerostin (SOST, UniProt: Q9BQB4) and Brorin (VWC2, UniProt: Q2TAL6). Examples of proteins in the BMP signaling pathway may also include Proprotein convertase subtilisin/kexin type 6 (PCSK6, UniProt: P29122) that regulates BMP signaling.

Small molecules, polynucleotides, polypeptides that enhance or inhibit BMP signaling exist or can be made with procedures known by those skilled in the art. Yanagita (2009) BioFactors 35(2):113-199 is a review article discussing BMP regulators (incorporated herein by reference). For example, dorsomorphin is a potent small molecule BMP antagonist (Hao et al. (2008) PLoS ONE 3(8):e2904, Yu et al. (2008) Nat. Chem. Biol. 4(1):33-41). Dorsomorphin is currently commercially available from several vendors. Dorsomorphin was reported to selectively inhibit the BMP receptors, type I: ALK2, ALK3 and ALK6 and thus “blocks BMP-mediated SMAD1/5/8 phosphorylation”. Dorsomorphin has preferential specificity toward inhibiting BMP versus TGF-beta and activin signaling. In published reports, dorsomorphin is characterized by low toxicity. Dorsomorphin can be delivered into skin to lower macro-environmental BMP signaling and create favorable conditions for hair growth to occur. A unique property of dorsomorphin is that it is a small molecule and is soluble in DMSO. DMSO is known to significantly facilitate trans-dermal delivery of small molecule drugs. This enhancing effect of DMSO on skin penetration can be used in non-invasive method of pharmacological modulation of dermal macro-environment. Treatment procedure thus consists of simply applying liquid form of dorsomorphin in DMSO onto the surface of intact skin. Dorsomorphin in DMSO can be made in form of cream that can be simply rubbed onto intact skin. Small molecule agonist and antagonists for other signaling pathways also exist and can be used to augment or inhibit BMP signaling. Interaction of these small molecules with pathways including, but not limited to, WNT, SHH and FGF will also have direct or indirect impact on BMP signaling thus serve as effective modulator of hair growth via methods disclosed in this invention.

Other types of BMP agonists or antagonists also exist. Yanagita (2009) BioFactors 35(2):113-199 is a review article discussing BMP regulators (incorporated herein by reference). Non-limiting examples include such as noggin, chordin, gremlin, sclerostin and follistatin. Representative sequences for these proteins include UniProt: Q13253 for noggin, UniProt: Q9H2X0 for chordin, UniProt: O60565 for gremlin, UniProt: Q9BQB4 for sclerostin, and UniProt: P19883 for follistatin. Noggin (UniProt: Q13253), for example, can be produced using methods described in, e.g. McMahon et al. (1998) Genes & Development 12:1438-52.

In some aspects, an agent that can augment or inhibit BMP signaling is a small molecule agonist or antagonist to a BMP agonist or antagonist. In one aspect, the small molecule is a noggin agonist. In another aspect, the small molecule is a noggin antagonist.

Examples of agents that can augment or inhibit BMP signaling also include, but are not limited to, polynucleotides that encode BMP proteins, encode polypeptides augmenting or inhibiting BMP signaling, or augmenting or inhibit expression of BMP proteins, or polypeptides augmenting or inhibiting BMP signaling. In some embodiments, the agent is small interference RNA (siRNA) or double strand RNA (dsRNA) that inhibits expression of proteins that augment or inhibit BMP signaling.

Examples of agents that can augment or inhibit BMP signaling may also include, but are not limited to, an isolated or recombinant BMP protein, or isolated or recombinant polypeptide enhancing or inhibiting BMP signaling. In some aspect, the agent further comprises a pharmaceutically acceptable carrier. In another aspect, the compositions contain carriers that modulate (controlled release) the release of the active agent when administered to a subject in need thereof.

Examples of polypeptide agents that augment or inhibit BMP signaling may also include, but are not limited to, antibodies or modified antibodies including, but not limited to, blocking fragments of antibodies, that activate, stabilize or inhibit proteins in the BMP signaling pathway or proteins modulating the BMP signaling pathway, thereby augmenting or inhibiting BMP signaling.

As used herein, the term “modulate” refers to an act by an agent to regulate, to control or to change certain characteristics of the BMP signaling. Examples of the agent may include, but are not limited to, proteins or polypeptides, DNA, RNA, siRNA, dsRNA or other polynucleotides, small molecules. The agent may also mean a temperature change, physical movement or stimulus or any other therapeutical or clinical means that alter the BMP signaling pathway. Without limitation, the object may mean a biochemical molecule or pathway, a biochemical activity, a medical condition or any other chemical, biochemical, physical or medical aspect of a subject. In one aspect, the term “modulate” means to enhance hair growth on the skin. In another aspect, the term “modulate” means to inhibit hair growth on the skin. In another aspect, the term “modulate” means to positively regulate BMP signaling. In yet another aspect, the term “modulate” means to negatively regulate BMP signaling.

The terms “facilitate”, “augment” and “enhance” as used herein refer to an increase of amount or activity of the target. In one aspect, they refer to activation of the BMP receptors and the downstream signaling, or activation of any downstream signaling without directly activating BMP. In another aspect, they refer to an increase of formation of new hairs on skin, in vivo or in vitro, or an increase of growth of existing hair.

The terms “inhibit” or “antagonize” intend mean an decrease of amount or activity of the target. In one aspect, they refer to decrease of activity of the BMP receptors and the downstream signaling, or decrease of any downstream signaling without directly interacting with BMP. In another aspect, they refer to an decrease of formation of new hairs on skin, in vivo or in vitro, or an reduction of growth of existing hair.

An “agonist”, as used herein, refers to a drug or other chemical that can bind a receptor on a cell to produce a physiologic reaction typical of a naturally occurring substance. The efficacy of an agonist may be positive, causing an increase in the receptor's activity or negative causing a decrease in the receptor's activity.

An “antagonist” refers to a type of receptor ligand or drug that does not provoke a biological response itself upon binding to the receptor, but blocks or dampens agonist-mediated responses. In pharmacology, antagonists have affinity but no efficacy for their cognate receptors and binding will disrupt the interaction and inhibit the function of an agonist or inverse agonist at receptors. Antagonists mediate their effects by binding to the active site or to allosteric sites on receptors or they may interact at unique binding sites not normally involved in the biological regulation of the receptor's activity. Antagonist activity may be reversible or irreversible depending on the longevity of the antagonist-receptor complex which in turn depends on the nature of antagonist receptor binding. The majority of drug antagonists achieve their potency by competing with endogenous ligands or substrates at structurally defined binding sites on receptors.

The term “hair growth” intends to include, but not limited to, the formation of new hair or growth of existing hair.

“Spironolactone” (IUPAC name: 7α-Acetylthio-3-oxo-17α-pregn-4-ene-21,17-carbolactone is marketed under the trade names Aldactone, Novo-Spiroton, Aldactazide, Spiractin, Spirotone, Verospiron or Berlactone) is a diuretic and is used as an antiandrogen. It is also used for treating hair loss in women, and can be used as a topical medication for treatment of male baldness.

“Minoxidil” (trade names Rogaine and Regaine; IUPAC name: 6-piperidin-1-ylpyrimidine-2,4-diamine 3-oxide) is a commercially available topical formulation that inhibits hair loss. is a vasodilator medication that is available over the counter for treatment of androgenic alopecia, among other baldness treatments.

“Finasteride” (IUPAC name N-(1,1-dimethylethyl)-3-oxo-(5α,17β)-4-azaandrost-1-ene-17-carboxamide) is a synthetic antiandrogen that acts by inhibiting type II 5-alpha reductase, the enzyme that converts testosterone to dihydrotestosterone (DHT). It is used to treat prostate cancer and is registered in many countries to treat adrogenetic alopecia or male pattern baldness. “Propecia” is a medicament containing finasteride as an active ingredient is commercially available from Merck & Co., Inc.

“Administration”, as used herein, refers to the delivery of a medication, such as the agent of the invention, which inhibits or augments the BMP signaling, to an appropriate location of the subject, where a therapeutic effect is achieved. Non-limiting examples include oral dosing, intracutaneous injection, direct application to target area proximal areas on the skin, or applied on a patch. Various physical and/or mechanical technologies are available to permit the sustained or immediate topical or transdermal administration of macromolecules (such as, peptides). Such technologies include iontophoresis (see for example Kalia et al., Adv. Drug Del. Rev. 56:619-58, 2004) sonophoresis, needle-less injection, and/or microstructured arrays (sometimes called microneedles; one particular example is the Microstructured Transdermal System (MTS) commercially available from 3M) (see, e.g., Alain et al. (2002) J. Control. Release 81:113-119; Santi et al. (1997) Pharm. Res., 14(1):63-66; Sebastien et al. (1998) J. Pharm. Sci. 87(8):922-925). Methods of making and using arrays of solid microneedles that can be inserted into the skin for transdermal delivery of peptides (such as cyclic CRF antagonists) are provided in Martanto et al. (2004) Pharm. Res. 21:947-52, and Am. Inst. Chem. Eng. 51:1599-607 (2005). In some examples, the delivery system includes a combination of systems, such as microneedles made of biocompatible and biodegradable polymers (Park et al. (2005) J. Control. Release 104:51-66). Laser systems have also been developed to ablate the stratum corneum from the epidermal layer (Lee et al. (2002) J. Pharm. Sci. 91(7): 1613-1626). The laser-ablated regions offer lower resistance to drug (peptide) diffusion than non-ablated skin. In one aspect, administration is topical administration as defined herein.

“Topical administration” refers to delivery of a medication by application to the skin. Non-limiting examples of topical administration include any methods described under the definition of “administration” pertaining to delivery of a medication to the skin.

“Interdermal administration” intends delivery of the active ingredient into the dermal layers of the skin, e.g., by use of microneedles or the like.

“Ablate” or “ablation” of tissue refers to surgical excision or amputation of part of organ or tissue. In one aspect, a mechanical surgical device can be used to excise a layer or part of a layer of the skin such as by tape stripping. In another aspect, laser is used to remove stratum corneum of epidermis to increase the permeability of the skin. The types of surgical devices and procedure, and the type and amount of laser used are known in the art. It should be understood although not always explicitly stated that ablation of tissue may be used prior to treatment as described herein.

A penetration or permeation enhancer refers to a chemical composition or mechanical/electrical device that can increase the transdermal drug delivery efficiency. In one aspect, a penetration or permeation enhancer is soluble in the formulation and act to reduce the barrier properties of human skin. The list of potential skin permeation enhancers is long, but can be broken down into three general categories: lipid disrupting agents (LDAs), solubility enhancers, and surfactants. LDAs are typically fatty acid-like molecules proposed to fluidize lipids in the human skin membrane. Solubility enhancers act by increasing the maximum concentration of drug in the formulation, thus creating a larger concentration gradient for diffusion. Surfactants are amphiphilic molecules capable of interacting with the polar and lipid groups in the skin (see e.g. Francoeur et al., Potts, Russell O. (1990) Pharm. Res. 7:621-7; U.S. Pat. No. 5,503,843).

A “composition” is intended to mean a combination of active agent, cell or population of cells and another compound or composition, inert (for example, a detectable agent or label or biocompatible scaffold) or active, such as a growth and/or differentiation factor.

A “pharmaceutical composition” is intended to include the combination of an active agent with a carrier, inert or active such as a biocompatible scaffold, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)). The term includes carriers that facilitate controlled release of the active agent as well as immediate release.

For topical use, the pharmaceutically acceptable carrier is suitable for manufacture of creams, ointments, jellies, gels, solutions, suspensions, etc. Such carriers are conventional in the art, e.g., for topical administration with polyethylene glycol (PEG). These formulations may optionally comprise additional pharmaceutically acceptable ingredients such as diluents, stabilizers, and/or adjuvants.

The pharmaceutically acceptable carrier facilitate immediate or controlled release of the active ingredient.

A “subject” of diagnosis or treatment is a composition, tissue or an animal, such as a mammal, including a human. Non-human animals subject to diagnosis or treatment include, for example, murine, such as rats, mice, canine, such as dogs, leporids, such as rabbits, bovine, simian, ovine, livestock, sport animals, and pets.

An “effective amount” is an amount sufficient to effect beneficial or desired results. An effective amount can be administered in one or more administrations, applications or dosages.

A “control” is an alternative subject or sample used in an experiment for comparison purpose. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype). Alternatively, a positive control is an agent exhibiting a desired biological response and a negative control is one that is known not to exhibit the desired biological response.

As used herein, the terms “treating,” “treatment” and the like are used herein to mean obtaining a desired pharmacologic and/or physiologic effect. The effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder, e.g., alopecia. As is understood by those skilled in the art, “treatment” can include systemic amelioration of the symptoms associated with the pathology and/or a delay in onset of symptoms such as hair loss.

Polynucleotides and Construction, Expression and Delivery

The terms “nucleic acid”, “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, small interference RNA (siRNA), double strand RNA (dsRNA), ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by ═HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on Nov. 26, 2007. Biologically equivalent polynucleotides are those having the specified percent homology and encoding a polypeptide having the same or similar biological activity.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

“RNA interference” (RNAi) refers to sequence-specific or gene specific suppression of gene expression (protein synthesis) that is mediated by short interfering RNA (siRNA).

“Short interfering RNA” (siRNA) refers to double-stranded RNA molecules, generally, from about 10 to about 30 nucleotides long that are capable of mediating RNA interference (RNAi)), or 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, or 29 nucleotides in length. As used herein, the term siRNA includes short hairpin RNAs (shRNAs).

“Double stranded RNA” (dsRNA) refer to double stranded RNA molecules that may be of any length and may be cleaved intracellularly into smaller RNA molecules, such as siRNA. In cells that have a competent interferon response, longer dsRNA, such as those longer than about 30 base pair in length, may trigger the interferon response. In other cells that do not have a competent interferon response, dsRNA may be used to trigger specific RNAi.

siRNA sequences can be designed by obtaining the target mRNA sequence and determining an appropriate siRNA complementary sequence. siRNAs of the invention are designed to interact with a target sequence, meaning they complement a target sequence sufficiently to hybridize to that sequence. An siRNA can be 100% identical to the target sequence. However, homology of the siRNA sequence to the target sequence can be less than 100% as long as the siRNA can hybridize to the target sequence. Thus, for example, the siRNA molecule can be at least 70%, 75%, 80%, 85%, 90%, 95%, 96%, 97%, 98%, 99% or 100% identical to the target sequence or the complement of the target sequence. Therefore, siRNA molecules with insertions, deletions or single point mutations relative to a target may also be used. The generation of several different siRNA sequences per target mRNA is recommended to allow screening for the optimal target sequence. A homology search, such as a BLAST search, should be performed to ensure that the siRNA sequence does not contain homology to any known mammalian gene.

In general, its preferable that the target sequence be located at least 100-200 nucleotides from the AUG initiation codon and at least 50-100 nucleotides away from the termination codon of the target mRNA (Duxbury (2004) J. Surgical Res. 117:339-344).

Researchers have determined that certain characteristics are common in siRNA molecules that effectively silence their target gene (Duxbury (2004) J. Surgical Res. 117:339-344; Ui-Tei et al. (2004) Nucl. Acids Res. 32:936-48). As a general guide, siRNAs that include one or more of the following conditions are particularly useful in gene silencing in mammalian cells: GC ratio of between 45-55%, no runs of more than 9 G/C residues, G/C at the 5′ end of the sense strand; NU at the 5′ end of the antisense strand; and at least 5 NU residues in the first 7 bases of the 5′ terminal of the antisense strand.

siRNA are, in general, from about 10 to about 30 nucleotides in length. For example, the siRNA can be 10-30 nucleotides long, 12-28 nucleotides long, 15-25 nucleotides long, 19-23 nucleotides long, or 21-23 nucleotides long. When an siRNA contains two strands of different lengths, the longer of the strands designates the length of the siRNA. In this situation, the unpaired nucleotides of the longer strand would form an overhang.

The term siRNA includes short hairpin RNAs (shRNAs). shRNAs comprise a single strand of RNA that forms a stem-loop structure, where the stem consists of the complementary sense and antisense strands that comprise a double-stranded siRNA, and the loop is a linker of varying size. The stem structure of shRNAs generally is from about 10 to about 30 nucleotides long. For example, the stem can be 10-30 nucleotides long, 12-28 nucleotides long, 15-25 nucleotides long, 19-23 nucleotides long, or 21-23 nucleotides long.

Tools to assist siRNA design are readily available to the public. For example, a computer-based siRNA design tool is available on the internet at www.dharmacon.com, last accessed on Nov. 26, 2007.

dsRNA and siRNA can be synthesized chemically or enzymatically in vitro as described in Micura (2002) Agnes Chem. Int. Ed. Emgl. 41:2265-2269; Betz (2003) Promega Notes 85:15-18; and Paddison and Hannon (2002) Cancer Cell. 2:17-23. Chemical synthesis can be performed via manual or automated methods, both of which are well known in the art as described in Micura (2002), supra. siRNA can also be endogenously expressed inside the cells in the form of shRNAs as described in Yu et al. (2002) Proc. Natl. Acad. Sci. USA 99:6047-6052; and McManus et al. (2002) RNA 8:842-850. Endogenous expression has been achieved using plasmid-based expression systems using small nuclear RNA promoters, such as RNA polymerase III U6 or H1, or RNA polymerase II U1 as described in Brummelkamp et al. (2002) Science 296:550-553 (2002); and Novarino et al. (2004) J. Neurosci. 24:5322-5330.

In vitro enzymatic dsRNA and siRNA synthesis can be performed using an RNA polymerase mediated process to produce individual sense and antisense strands that are annealed in vitro prior to delivery into the cells of choice as describe in Fire et al. (1998) Nature 391:806-811; Donze and Picard (2002) Nucl. Acids Res. 30(10):e46; Yu et al. (2002); and Shim et al. (2002) J. Biol. Chem. 277:30413-30416. Several manufacturers (Promega, Ambion, New England Biolabs, and Stragene) produce transcription kits useful in performing the in vitro synthesis.

In vitro synthesis of siRNA can be achieved, for example, by using a pair of short, duplex oligonucleotides that contain T7 RNA polymerase promoters upstream of the sense and antisense RNA sequences as the DNA template. Each oligonucleotide of the duplex is a separate template for the synthesis of one strand of the siRNA. The separate short RNA strands that are synthesized are then annealed to form siRNA as described in Protocols and Applications, Chapter 2: RNA interference, Promega Corporation, (2005).

In vitro synthesis of dsRNA can be achieved, for example, by using a T7 RNA polymerase promoter at the 5′-ends of both DNA target sequence strands. This is accomplished by using separate DNA templates, each containing the target sequence in a different orientation relative to the T7 promoter, transcribed in two separate reactions. The resulting transcripts are mixed and annealed post-transcriptionally. DNA templates used in this reaction can be created by PCR or by using two linearized plasmid templates, each containing the T7 polymerase promoter at a different end of the target sequence. Protocols and Applications, Chapter 2: RNA interference, Promega Corporation, (2005).

In order to express the proteins described herein, delivery of nucleic acid sequences encoding the gene of interest can be delivered by several techniques. Examples of which include viral technologies (e.g. retroviral vectors, adenovirus vectors, adeno-associated virus vectors, alphavirus vectors and the like) and non-viral technologies (e.g. DNA/liposome complexes, micelles and targeted viral protein-DNA complexes) as described herein. Once inside the cell of interest, expression of the transgene can be under the control of ubiquitous promoters (e.g. EF-1α) or tissue specific promoters (e.g. keratin 14 promoter (Plikus (2004) J. Pathol. 164:1099-1144; Calcium Calmodulin kinase 2 (CaMKI) promoter, NSE promoter and human Thy-1 promoter). Alternatively expression levels may controlled by use of an inducible promoter system (e.g. Tet on/off promoter) as described in Wiznerowicz et al. (2005) Stem Cells 77:8957-8961.

Non-limiting examples of promoters include, but are not limited to, the cytomegalovirus (CMV) promoter (Kaplitt et al. (1994) Nat. Genet. 8:148-154), CMV/human β3-globin promoter (Mandel et al. (1998) J. Neurosci. 18:4271-4284), NCX1 promoter, αMHC promoter, MLC2v promoter, GFAP promoter (Xu et al. (2001) Gene Ther., 8:1323-1332), the 1.8-kb neuron-specific enolase (NSE) promoter (Klein et al. (1998) Exp. Neurol. 150:183-194), chicken beta actin (CBA) promoter (Miyazaki (1989) Gene 79:269-277) and the β-glucuronidase (GUSB) promoter (Shipley et al. (1991) Genetics 10:1009-1018), the human serum albumin promoter, the alpha-1-antitrypsin promoter. To improve expression, other regulatory elements may additionally be operably linked to the transgene, such as, e.g., enhancer elements, the Woodchuck Hepatitis Virus Post-Regulatory Element (WPRE) (Donello et al. (1998) J. Virol. 72: 5085-5092) or the bovine growth hormone (BGH) polyadenylation site.

A “gene product” or alternatively a “gene expression product” refers to the amino acid (e.g., peptide or polypeptide) generated when a gene is transcribed and translated.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells or cardiomyocytes. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarrays” and similar technologies are know in the art. Examples of such include, but are not limited to, LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarrying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu. Rev. Biomed. Eng. 4:129-153. Examples of “gene chips” or a “microarrays” are also described in U.S. Patent Publ. Nos.: 2007-0111322, 2007-0099198, 2007-0084997, 2007-0059769 and 2007-0059765 and U.S. Pat. Nos. 7,138,506, 7,070,740, and 6,989,267.

In one aspect, “gene chips” or “microarrays” containing probes or primers homologous to a polynucleotide, polypeptide or antibody described herein are prepared. A suitable sample is obtained from the patient, extraction of genomic DNA, RNA, protein or any combination thereof is conducted and amplified if necessary. The sample is contacted to the gene chip or microarray panel under conditions suitable for hybridization of the gene(s) or gene product(s) of interest to the probe(s) or primer(s) contained on the gene chip or microarray. The probes or primers may be detectably labeled thereby identifying the gene(s) of interest. Alternatively, a chemical or biological reaction may be used to identify the probes or primers which hybridized with the DNA or RNA of the gene(s) of interest. The genotypes or phenotype of the patient is then determined with the aid of the aforementioned apparatus and methods.

Other non-limiting examples of a solid phase support include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the coupled molecule is capable of binding to a polynucleotide, polypeptide or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding antibody or antigen, or will be able to ascertain the same by use of routine experimentation.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. A eukaryotic host, including, for example, yeast, higher plant, insect and mammalian cells, or alternatively from a prokaryotic cells as described above. Non-limiting examples include simian, bovine, porcine, murine, rats, avian, reptilian and human.

“Prokaryotic cells” that usually lack a nucleus or any other membrane-bound organelles and are divided into two domains, bacteria and archaea. Additionally, instead of having chromosomal DNA, these cells' genetic information is in a circular loop called a plasmid. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to bacillus bacteria, E. coli bacterium, and Salmonella bacterium.

A “transgenic animal”, as used herein, refers to a non-human animal comprising an expression cassette, or a heterologous nucleic acid stably integrated into the animal genome, which expression cassette comprises a polynucleotide encoding a BMP protein, including but not limited to BMP1, BMP2, BMP3, BMP4, BMP5, BMP6, BMP7, BMP8a, BMP8b, BMP10 and BMP15, under control of a skin-specific promoter, such as the keratin 14 promoter. The heterologous nucleic acid is introduced into the animal by genetic engineering techniques, such as by trangenic techniques known by those skilled in the art. In another aspect, the expression cassette comprises a polynucleotide encoding a BMP antagonist, such as noggin, chordin, gremlin, sclerostin and follistatin. More details of constructing the expression cassette and transgenic animal are described in Pilkus et al. (2004) Am. J. Pathol. 164:1099-114.

The term “expression cassette” or “transgenic gene construct” refers to a nucleic acid molecule, e.g., a vector, containing the subject gene, e.g., BMP1, BMP2, BMP3, BMP4, BMP5, BMP6, BMP7, BMP8a, BMP8b, BMP10 and BMP15, operably linked in a manner capable of expressing the gene in a host cell. The expression cassette or gene construct can be introduced into a non-human animal cell by nucleic acid-mediated gene transfer by methods known to those skilled in the art.

In certain aspects, the invention is related to an isolated or recombinant BMP protein, polypeptide BMP agonist or antagonist, examples of which are described herein as well as in Yanagita (2009) BioFactors 35(2):113-119. Yanagita (2009) supra., reports BMP antagonists and agonists known in the art. Agonists include repulsive guidance molecule (RGMA), DRAGON (RGMB), hemojuvelin, kielin/chordin-like protein (KCP), and Crossveinless 2 (Cv2). Antagonists include chordin, noggin, the eight-membered rings Dan family, the nine-membered ring Tsg family and Crim1. Also encompassed by this invention are polypeptides having at least 80% sequence identify, or alternatively 85% sequence identify, or alternatively 90% sequence identity, or alternatively 95% sequence identify, to these polypeptide agonists and antagonists.

Polypeptides of the invention can be prepared by expressing polynucleotides encoding the polypeptide sequences of this invention in an appropriate host cell. This can be accomplished by methods of recombinant DNA technology known to those skilled in the art. Accordingly, this invention also provides methods for recombinantly producing the polypeptides of this invention in a eukaryotic or prokaryotic host cells. The proteins and polypeptides of this invention also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this invention also provides a process for chemically synthesizing the proteins of this invention by providing the sequence of the protein and reagents, such as amino acids and enzymes and linking together the amino acids in the proper orientation and linear sequence.

It is known to those skilled in the art that modifications can be made to any peptide to provide it with altered properties. Polypeptides of the invention can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that α-helical secondary structure or random secondary structure is preferred.

In a further embodiment, subunits of polypeptides that confer useful chemical and structural properties will be chosen. For example, peptides comprising D-amino acids may be resistant to L-amino acid-specific proteases in vivo. Modified compounds with D-amino acids may be synthesized with the amino acids aligned in reverse order to produce the peptides of the invention as retro-inverso peptides. In addition, the present invention envisions preparing peptides that have better defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂NH—R₂, where R₁, and R₂ are amino acid residues or sequences. A reduced peptide bond may be introduced as a dipeptide subunit. Such a molecule would be resistant to peptide bond hydrolysis, e.g., protease activity. Such molecules would provide ligands with unique function and activity, such as extended half-lives in vivo due to resistance to metabolic breakdown, or protease activity. Furthermore, it is well known that in certain systems constrained peptides show enhanced functional activity (Hruby (1982) Life Sciences 31:189-199 and Hruby et al. (1990) Biochem J. 268:249-262); the present invention provides a method to produce a constrained peptide that incorporates random sequences at all other positions.

The following non-classical amino acids may be incorporated in the peptides of the invention in order to introduce particular conformational motifs: 1,2,3,4-tetrahydroisoquinoline-3-carboxylate (Kazrnierski et al. (1991) J. Am. Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski and Hruby (1991) Tetrahedron Lett. 32(41):5769-5772); 2-aminotetrahydronaphthalene-2-carboxylic acid (Landis (1989) Ph.D. Thesis, University of Arizona); hydroxy-1,2,3,4-tetrahydroisoquinoline-3-carboxylate (Miyake et al. (1989) J. Takeda Res. Labs. 43:53-76) histidine isoquinoline carboxylic acid (Zechel et al. (1991) Int. J. Pep. Protein Res. 38(2):131-138); and HIC (histidine cyclic urea), (Dharanipragada et al. (1993) Int. J. Pep. Protein Res. 42(1):68-77) and (Dharanipragada et al. (1992) Acta. Crystallogr. C. 48:1239-1241).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog (Kemp et al. (1985) J. Org. Chem. 50:5834-5838); β-sheet inducing analogs (Kemp et al. (1988) Tetrahedron Lett. 29:5081-5082); β-turn inducing analogs (Kemp et al. (1988) Tetrahedron Lett. 29:5057-5060); α-helix inducing analogs (Kemp et al. (1988) Tetrahedron Lett. 29:4935-4938); α-turn inducing analogs (Kemp et al. (1989) J. Org. Chem. 54:109:115); analogs provided by the following references: Nagai and Sato (1985) Tetrahedron Lett. 26:647-650; and DiMaio et al. (1989) J. Chem. Soc. Perkin Trans. p. 1687; a Gly-Ala turn analog (Kahn et al. (1989) Tetrahedron Lett. 30:2317); amide bond isostere (Clones et al. (1988) Tetrahedron Lett. 29:3853-3856); tetrazole (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am. Chem. Sci. 112:323-333 and Garvey et al. (1990) J. Org. Chem. 56:436. Conformationally restricted mimetics of beta turns and beta bulges, and peptides containing them, are described in U.S. Pat. No. 5,440,013, issued Aug. 8, 1995 to Kahn.

It is known to those skilled in the art that modifications can be made to any peptide by substituting one or more amino acids with one or more functionally equivalent amino acids that does not alter the biological function of the peptide. In one aspect, the amino acid that is substituted by an amino acid that possesses similar intrinsic properties including, but not limited to, hydrophobicity, size, or charge. Methods used to determine the appropriate amino acid to be substituted and for which amino acid are know to one of skill in the art. Non-limiting examples include empirical substitution models as described by Dahoff et al. (1978) In Atlas of Protein Sequence and Structure Vol. 5 suppl. 2 (ed. M. O. Dayhoff), pp. 345-352. National Biomedical Research Foundation, Washington D.C.; PAM matrices including Dayhoff matrices (Dahoff et al. (1978), supra, or JTT matrices as described by Jones et al. (1992) Comput. Appl. Biosci. 8:275-282 and Gonnet et al. (1992) Science 256:1443-1145; the empirical model described by Adach and Hasegawa (1996) J. Mol. Evol. 42:459-468; the block substitution matrices (BLOSUM) as described by Henikoff and Henikoff (1992) Proc. Natl. Acad. Sci. USA 89:10915-10919; Poisson models as described by Nei (1987) Molecular Evolutionary Genetics. Columbia University Press, New York; and the Maximum Likelihood (ML) Method as described by Müller et al. (2002) Mol. Biol. Evol. 19:8-13.

### Polypeptide Conjugates

The polypeptides and polypeptide complexes of the invention can be used in a variety of formulations, which may vary depending on the intended use. For example, one or more can be covalently or non-covalently linked (complexed) to various other molecules, the nature of which may vary depending on the particular purpose. For example, a peptide of the invention can be covalently or non-covalently complexed to a macromolecular carrier, including, but not limited to, natural and synthetic polymers, proteins, polysaccharides, polypeptides (amino acids), polyvinyl alcohol, polyvinyl pyrrolidone, and lipids. A peptide can be conjugated to a fatty acid, for introduction into a liposome, see U.S. Pat. No. 5,837,249. A peptide of the invention can be complexed covalently or non-covalently with a solid support, a variety of which are known in the art and described herein. An antigenic peptide epitope of the invention can be associated with an antigen-presenting matrix such as an MHC complex with or without co-stimulatory molecules.

Examples of protein carriers include, but are not limited to, superantigens, serum albumin, tetanus toxoid, ovalbumin, thyroglobulin, myoglobulin, and immunoglobulin.

Peptide-protein carrier polymers may be formed using conventional cross-linking agents such as carbodimides. Examples of carbodimides are 1-cyclohexyl-3-(2-morpholinyl-(4-ethyl)carbodiimide (CMC), 1-ethyl-3-(3-dimethyaminopropyl)carbodiimide (EDC) and 1-ethyl-3-(4-azonia-44-dimethylpentyl)carbodiimide.

Examples of other suitable cross-linking agents are cyanogen bromide, glutaraldehyde and succinic anhydride. In general, any of a number of homo-bifunctional agents including a homo-bifunctional aldehyde, a homo-bifunctional epoxide, a homo-bifunctional imido-ester, a homo-bifunctional N-hydroxysuccinimide ester, a homo-bifunctional maleimide, a homo-bifunctional alkyl halide, a homo-bifunctional pyridyl disulfide, a homo-bifunctional aryl halide, a homo-bifunctional hydrazide, a homo-bifunctional diazonium derivative and a homo-bifunctional photoreactive compound may be used. Also included are hetero-bifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homo-bifunctional cross-linking agents include the bifunctional N-hydroxysuccinimide esters dithiobis(succinimidylpropionate), disuccinimidyl suberate, and disuccinimidyl tartrate; the bifunctional imido-esters dimethyl adipimidate, dimethyl pimelimidate, and dimethyl suberimidate; the bifunctional sulfhydryl-reactive crosslinkers 1,4-di-[3′-(2′-pyridyldithio)propionamido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1 N′-ethylene-bis(iodoacetamide), N1 N′-hexamethylene-bis(iodoacetamide), N1 N′-undecamethylene-bis(iodoacetamide), as well as benzylhalides and halomustards, such as a1a′-diiodo-p-xylene sulfonic acid and tri(2-chloroethyl)amine, respectively.

Examples of common hetero-bifunctional cross-linking agents that may be used to effect the conjugation of proteins to peptides include, but are not limited to, SMCC (succinimidyl-4-(N-maleimidomethyl)cyclohexane-1-carboxylate), MBS (m-maleimidobenzoyl-N-hydroxysuccinimide ester), STAB (N-succinimidyl(4-iodoacteyl)aminobenzoate), SMPB (succinimidyl-4-(p-maleimidophenyl)butyrate), GMBS (N-(γ-maleimidobutyryloxy)succinimide ester), MPBH (4-(4-N-maleimidopohenyl)butyric acid hydrazide), M2C2H (4-(N-maleimidomethyl)cyclohexane-1-carboxyl-hydrazide), SMPT (succinimidyloxycarbonyl-α-methyl-α-(2-pyridyldithio)toluene), and SPDP (N-succinimidyl 3-(2-pyridyldithio)propionate).

Cross-linking may be accomplished by coupling a carbonyl group to an amine group or to a hydrazide group by reductive amination.

Peptides of the invention also may be formulated as non-covalent attachment of monomers through ionic, adsorptive, or biospecific interactions. Complexes of peptides with highly positively or negatively charged molecules may be done through salt bridge formation under low ionic strength environments, such as in deionized water. Large complexes can be created using charged polymers such as poly-(L-glutamic acid) or poly-(L-lysine) which contain numerous negative and positive charges, respectively. Adsorption of peptides may be done to surfaces such as microparticle latex beads or to other hydrophobic polymers, forming non-covalently associated peptide-superantigen complexes effectively mimicking cross-linked or chemically polymerized protein. Finally, peptides may be non-covalently linked through the use of biospecific interactions between other molecules. For instance, utilization of the strong affinity of biotin for proteins such as avidin or streptavidin or their derivatives could be used to form peptide complexes. These biotin-binding proteins contain four binding sites that can interact with biotin in solution or be covalently attached to another molecule. (See Wilchek (1988) Anal. Biochem. 171:1-32). Peptides can be modified to possess biotin groups using common biotinylation reagents such as the N-hydroxysuccinimidyl ester of D-biotin (NHS-biotin) which reacts with available amine groups on the protein. Biotinylated peptides then can be incubated with avidin or streptavidin to create large complexes. The molecular mass of such polymers can be regulated through careful control of the molar ratio of biotinylated peptide to avidin or streptavidin.

Also provided by this application are the peptides and polypeptides described herein conjugated to a label, e.g., a fluorescent or bioluminescent label, for use in the diagnostic methods. For example, detectably labeled peptides and polypeptides can be bound to a column and used for the detection and purification of antibodies. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in Haugland, Richard P. (1996) Molecular Probes Handbook.

The polypeptides of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to, Freund's Complete Adjuvant, Freund's Incomplete Adjuvant and mineral salts.

### Therapeutic Antibody Compositions

This invention also provides an antibody capable of modulating BMP signaling by forming a complex with a BMP protein, a protein or polypeptide in the BMP signaling pathway, or a protein or polypeptide, such as a BMP agonist or antagonist, that modulates BMP signaling. In some embodiments, the antibody is a modified polypeptide of the antibody as described herein. In some embodiments, the antibody is a blocking fragment of the antibody. These antibodies can target intracellular or extracellular signaling elements and therefore either promote or antagonize BMP function. The BMP signaling pathway is described in Anderson et al. (2008) Nature Chem. Bio. 4(2):15-16. Antagonist include, for example noggin and/or chordin proteins.

The term “antibody” includes polyclonal antibodies and monoclonal antibodies, antibody fragments, as well as derivatives thereof (described above). The antibodies include, but are not limited to mouse, rat, and rabbit or human antibodies. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. The antibodies are also useful to identify and purify therapeutic polypeptides.

This invention also provides an antibody-peptide complex comprising antibodies described above and a polypeptide that specifically binds to the antibody. In one aspect the polypeptide is the polypeptide against which the antibody was raised. In one aspect the antibody-peptide complex is an isolated complex. In a further aspect, the antibody of the complex is, but not limited to, a polyclonal antibody, a monoclonal antibody, a humanized antibody or an antibody derivative described herein. Either or both of the antibody or peptide of the antibody-peptide complex can be detectably labeled. In one aspect, the antibody-peptide complex of the invention can be used as a control or reference sample in diagnostic or screening assays.

Polyclonal antibodies of the invention can be generated using conventional techniques known in the art and are well-described in the literature. Several methodologies exist for production of polyclonal antibodies. For example, polyclonal antibodies are typically produced by immunization of a suitable mammal such as, but not limited to, chickens, goats, guinea pigs, hamsters, horses, mice, rats, and rabbits. An antigen is injected into the mammal, which induces the B-lymphocytes to produce IgG immunoglobulins specific for the antigen. This IgG is purified from the mammals serum. Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antigen depot, which allows for a slow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods described in U.S. Pat. Nos. 7,279,559; 7,119,179; 7,060,800; 6,709,659; 6,656,746; 6,322,788; 5,686,073; and 5,670,153.

The monoclonal antibodies of the invention can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U397, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O) or the like, or heteromyelomas, fusion products thereof, or any cell or fusion cell derived therefrom, or any other suitable cell line as known in the art (see, e.g., www.atcc.org, www.lifetech.com., last accessed on Nov. 26, 2007, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing-heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods.

In one embodiment, the antibodies described herein can be generated using a Multiple Antigenic Peptide (MAP) system. The MAP system utilizes a peptidyl core of three or seven radially branched lysine residues, on to which the antigen peptides of interest can be built using standard solid-phase chemistry. The lysine core yields the MAP bearing about 4 to 8 copies of the peptide epitope depending on the inner core that generally accounts for less than 10% of total molecular weight. The MAP system does not require a carrier protein for conjugation. The high molar ratio and dense packing of multiple copies of the antigenic epitope in a MAP has been shown to produce strong immunogenic response. This method is described in U.S. Pat. No. 5,229,490 and is herein incorporated by reference in its entirety.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1977) Microbiol. Immunol. 41:901-907 (1997); Sandhu et al. (1996) Crit. Rev. Biotechnol. 16:95-118; Eren et al. (1998) Immunol. 93:154-161 that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al., Proc. Natl. Acad. Sci. USA (1996) 93:7843-7848); gel microdroplet and flow cytometry (Powell et al. (1990) Biotechnol. 8:333-337; One Cell Systems, (Cambridge, Mass.); Gray et al. (1995) J. 1 mm. Meth. 182:155-163; and Kenny et al. (1995) Bio. Technol. 13:787-790); B-cell selection (Steenbakkers et al. (1994) Molec. Biol. Reports 19:125-134.

Antibody derivatives of the present invention can also be prepared by delivering a polynucleotide encoding an antibody of this invention to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody derivative” includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fc-mediated cellular toxicity, and glycoproteins so generated.

Antibody derivatives also can be prepared by delivering a polynucleotide of this invention to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and reference cited therein. Thus, antibodies of the present invention can also be produced using transgenic plants, according to know methods.

Antibody derivatives also can be produced, for example, by adding exogenous sequences to modify immunogenicity or reduce, enhance or modify binding, affinity, on-rate, off-rate, avidity, specificity, half-life, or any other suitable characteristic. Generally part or all of the non-human or human CDR sequences are maintained while the non-human sequences of the variable and constant regions are replaced with human or other amino acids.

In general, the CDR residues are directly and most substantially involved in influencing antigen binding. Humanization or engineering of antibodies of the present invention can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel et al. (2000) Infection and Immunity April 2000:1820-1826; Gallo et al. (2000) European J. of Immun. 30:534-540; Green (1999) J. of Immun. Methods 231:11-23; Yang et al. (1999A) J. of Leukocyte Biology 66:401-410; Yang (1999B) Cancer Research 59(6):1236-1243; Jakobovits (1998) Advanced Drug Delivery Reviews 31:33-42; Green and Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies of this invention also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies of this invention can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species antibodies. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological Interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498.

The term “antibody derivative” also includes “diabodies” which are small antibody fragments with two antigen-binding sites, wherein fragments comprise a heavy chain variable domain (VH) connected to a light chain variable domain (VL) in the same polypeptide chain. (See for example, EP 404,097; WO 93/11161; and Hollinger et al., (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al. which discloses antibody variants that have one or more amino acids inserted into a hypervariable region of the parent antibody and a binding affinity for a target antigen which is at least about two fold stronger than the binding affinity of the parent antibody for the antigen.)

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Fd segments (V_{H}—C_{H}1-VH—C_{H}1) which form a pair of antigen binding regions. Linear antibodies can be bispecific or monospecific.

The antibodies of this invention can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies of the present invention include naturally purified products, products of chemical synthetic procedures, and products produced by recombinant techniques from a eukaryotic host, including, for example, yeast, higher plant, insect and mammalian cells, or alternatively from a prokaryotic cells as described above.

If a monoclonal antibody being tested binds with protein or polypeptide, then the antibody being tested and the antibodies provided by the hybridomas of this invention are equivalent. It also is possible to determine without undue experimentation, whether an antibody has the same specificity as the monoclonal antibody of this invention by determining whether the antibody being tested prevents a monoclonal antibody of this invention from binding the protein or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653 or Spira et al. (1984) J. Immunol. Methods 74:307.

The isolation of other hybridomas secreting monoclonal antibodies with the specificity of the monoclonal antibodies of the invention can also be accomplished by one of ordinary skill in the art by producing anti-idiotypic antibodies. Herlyn et al. (1986) Science 232:100. An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

In some aspects of this invention, it will be useful to detectably or therapeutically label the antibody. Suitable labels are described supra. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

The coupling of antibodies to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

Antibodies can be labeled with a detectable moiety such as a radioactive atom, a chromophore, a fluorophore, or the like. Such labeled antibodies can be used for diagnostic techniques, either in vivo, or in an isolated test sample. Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re)^{,} and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from Chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

The antibodies of the invention also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

### Pharmaceutical Compositions

In one aspect, the invention provides compositions used in the methods. In some embodiments, the compositions are small molecules that enhance or inhibit BMP signaling. In some embodiments, the compositions are polynucleotides that encode BMP proteins, encode polypeptides enhancing or inhibiting BMP signaling, or enhance or inhibit expression of BMP proteins, or polypeptides enhancing or inhibiting BMP signaling. In some embodiments, the compositions are isolated or recombinant BMP proteins, or isolated or recombinant polypeptides enhancing or inhibiting BMP signaling. Examples of each of these agents are described in this application and are the active agents in the pharmaceutical compositions.

In some aspect, the composition further comprises a pharmaceutically acceptable carrier, e.g., DMSO. In another aspect, the compositions contain carriers that modulate (controlled release) the release of the active agent when administered to a subject in need thereof. For example, the carriers can also include transdermal

The pharmaceutical compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Formulations may optionally contain stabilizers, pH modifiers, surfactants, bioavailability modifiers and combinations of these.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

The compositions of this invention are formulated for pharmaceutical administration to a mammal, preferably a human being. Such pharmaceutical compositions of the invention may be administered in a variety of ways, preferably topically or intradermally.

Pharmaceutically acceptable excipients and carriers and dosage forms are generally known to those skilled in the art and are included in the invention. It should be understood that a specific dosage and treatment regimen for any particular patient will depend upon a variety of factors, including the activity of the specific antidote employed, the age, body weight, general health, sex and diet, renal and hepatic function of the patient, and the time of administration, rate of excretion, drug combination, judgment of the treating physician or veterinarian and severity of the particular disease being treated.

For prophylactic administration, the compound can be administered to a patient at risk of developing one of the previously described conditions. For example, prophylactic administration can be applied to avoid the onset of symptoms in a patient diagnosed with the underlying disorder such as alopecia or a genetic predisposition to alocpecia.

The amount of compound administered will depend upon a variety of factors, including, for example, the particular indication being treated, the mode of administration, whether the desired benefit is prophylactic or therapeutic, the severity of the indication being treated and the age and weight of the patient, and the bioavailability of the particular active compound. Determination of an effective dosage is well within the capabilities of those skilled in the art.

Effective dosages can be estimated initially from in vitro assays. For example, an initial dosage for use in animals can be formulated to achieve a local (topical) or circulating blood or serum concentration of active compound that is at or above an IC₅₀ of the particular compound as measured in as in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, and the references cited therein.

Initial dosages can also be estimated from in vivo data, such as animal models. Animal models useful for testing the efficacy of compounds to treat or prevent the various diseases described above are well-known in the art. Ordinarily skilled artisans can routinely adapt such information to determine dosages suitable for human administration.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) which are sufficient to maintain therapeutic or prophylactic effect. For example, the compounds can be administered once per week, several times per week (e.g., every other day), once per day, or multiple times per day, depending upon, among other things, the mode of administration, the specific indication being treated, and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Preferably, the compound(s) will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

One aspect of the invention comprises small molecules that enhance or inhibit BMP signaling. Small molecule agonist and antagonists for other signaling pathways exist. Interaction of these small molecules with pathways including, but not limited to, WNT, SHH and FGF will also have direct or indirect impact on BMP signaling thus serve as effective modulator of hair growth via methods disclosed in this invention. Non-limiting examples include the proteins noggin, chordin, and dorsomorphin, a small molecule inhibitor of BMP signaling. For more details of the mechanism and composition of dorsomorphin, see Hao et al. (2008) PLoS ONE, 3(8):e2904 and Yu et al. (2008) Nat Chem. Biol. 4(1):33-41. Dorsomorphin was reported to selectively inhibit the BMP receptors, type I: ALK2, ALK3 and ALK6 and thus “blocks BMP-mediated SMAD1/5/8 phosphorylation”. Dorsomorphin has preferential specificity toward inhibiting BMP versus TGF-beta and activin signaling. In published reports, Dorsomorphin is characterized by low toxicity. Dorsomorphin is currently commercially available from several vendors. Dorsomorphin can be delivered into skin to lower macro-environmental BMP signaling and create favorable conditions for hair growth to occur. A unique property of Dorsomorphin is that it is a small molecule and is soluble in DMSO. DMSO is known to significantly facilitate trans-dermal delivery of small molecule drugs. This enhancing effect of DMSO on skin penetration can be used in non-invasive method of pharmacological modulation of dermal macro-environment. Treatment procedure thus consists of simply applying liquid form of Dorsomorphin in DMSO onto the surface of intact skin. Dorsomorphin in DMSO can be made in form of cream. Cream can be simply rubbed onto intact skin. Additional agents can be co-formulated or delivered concomitantly or sequentially with the above noted agents, e.g., minoxidil. The formulations can be for immediate or controlled release of the active ingredients.

Another aspect of the invention comprises polynucleotides that encode BMP proteins, encode polypeptides enhancing or inhibiting BMP signaling, or enhance or inhibit expression of BMP proteins, or polypeptides enhancing or inhibiting BMP signaling. Examples of such polynucleotides include, but are not limited to, nucleotides encoding BMP proteins, ligands to BMP proteins and proteins in the BMP signal pathway and polypeptides homologous or having at least 80%, or alternatively, at least 85%, or alternatively at least 90%, or alternatively at least 95%, or alternatively at least 98% sequence identity to these proteins. Non-limiting examples also include siRNA that interferences with expression of such polypeptides. Additional agents can be co-formulated or delivered concomitantly or sequentially with the above noted agents, e.g., minoxidil. The formulations can be for immediate or controlled release of the active ingredients.

Another aspect of the invention comprises isolated or recombinant BMP proteins, or isolated or recombinant polypeptides enhancing or inhibiting BMP signaling. In some aspect, the composition further comprises a pharmaceutically acceptable carrier. The polypeptides and polypeptide complexes of the invention can be used in a variety of formulations, which may vary depending on the intended use. For example, one or more can be covalently or non-covalently linked (complexed) to various other molecules, the nature of which may vary depending on the particular purpose. For example, a peptide of the invention can be covalently or non-covalently complexed to a macromolecular carrier, including, but not limited to, natural and synthetic polymers, proteins, polysaccharides, polypeptides (amino acids), polyvinyl alcohol, polyvinyl pyrrolidone, and lipids. A peptide can be conjugated to a fatty acid, for introduction into a liposome, see U.S. Pat. No. 5,837,249. A peptide of the invention can be complexed covalently or non-covalently with a solid support, a variety of which are known in the art and described herein. An antigenic peptide epitope of the invention can be associated with an antigen-presenting matrix such as an MHC complex with or without co-stimulatory molecules. Examples of protein carriers include, but are not limited to, superantigens, serum albumin, tetanus toxoid, ovalbumin, thyroglobulin, myoglobulin, and immunoglobulin.

Polypeptides may also be formulated as non-covalent attachment of monomers through ionic, adsorptive, or biospecific interactions. Complexes of peptides with highly positively or negatively charged molecules may be done through salt bridge formation under low ionic strength environments, such as in deionized water. Large complexes can be created using charged polymers such as poly-(L-glutamic acid) or poly-(L-lysine) which contain numerous negative and positive charges, respectively. Adsorption of peptides may be done to surfaces such as microparticle latex beads or to other hydrophobic polymers, forming non-covalently associated peptide-superantigen complexes effectively mimicking cross-linked or chemically polymerized protein. Finally, peptides may be non-covalently linked through the use of biospecific interactions between other molecules. For instance, utilization of the strong affinity of biotin for proteins such as avidin or streptavidin or their derivatives could be used to form peptide complexes. These biotin-binding proteins contain four binding sites that can interact with biotin in solution or be covalently attached to another molecule. (See Wilchek (1988) Anal. Biochem. 171:1-32). Peptides can be modified to possess biotin groups using common biotinylation reagents such as the N-hydroxysuccinimidyl ester of D-biotin (NHS-biotin) which reacts with available amine groups on the protein.

The polypeptides also can be combined with various liquid phase carriers, such as sterile or aqueous solutions, pharmaceutically acceptable carriers, suspensions and emulsions for immediate or controlled release. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to, Freund's Complete Adjuvant, Freund's Incomplete Adjuvant and mineral salts.

### Kits

An aspect of the invention provides a kit for inhibiting hair growth in a tissue having a hair follicle, comprising an effective amount of an agent that augments BMP in a pharmaceutically acceptable carrier and instructions for use in inhibiting hair growth. Another aspect of the invention provides a kit for augmenting or promoting hair growth comprising an effective amount of an agent that inhibits BMP in a pharmaceutically acceptable carrier and instructions for use in augmenting or promoting hair growth. Additional agents can be co-formulated or delivered concomitantly or sequentially with the above noted agents, e.g., minoxidil and provided in the kits. The formulations can be for immediate or controlled release of the active ingredients.

In some embodiments, the pharmaceutically acceptable carrier in the kits is suitable for topical administration of the agent. Additional agents can be co-formulated or delivered concomitantly or sequentially with the above noted agents, e.g., minoxidil. The formulations can be for immediate or controlled release of the active ingredients.

In some embodiments, the pharmaceutically acceptable carrier further comprises a penetration or permeation enhancer.

Also provided are kits for administration of the compounds for treatment of disorders as described herein. Kits may further comprise suitable packaging and/or instructions for use of the compound. Kits may also comprise a means for the delivery of the at least one agonist or antagonist and instructions for administration. Alternatively, the kit provides the compound and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch, or inhalant.

The kits may include other therapeutic compounds for use in conjunction with the compounds described herein. These compounds can be provided in a separate form or mixed with the compounds of the present invention.

The kits will include appropriate instructions for preparation and administration of the composition, side effects of the compositions, and any other relevant information. The instructions can be in any suitable format, including, but not limited to, printed matter, videotape, computer readable disk, or optical disc.

In another aspect of the invention, kits for treating an individual who suffers from or is susceptible to the conditions described herein are provided, comprising a container comprising a dosage amount of a composition, as disclosed herein, and instructions for use. The container can be any of those known in the art and appropriate for storage and delivery of oral, intravenous, topical, rectal, urethral, or inhaled formulations.

Kits may also be provided that contain sufficient dosages of the effective composition or compound to provide effective treatment for an individual for an extended period, such as a week, 2 weeks, 3, weeks, 4 weeks, 6 weeks, or 8 weeks or more.

### Therapeutic, Diagnostic and Screening Utilities

This invention provides the follow therapeutic, diagnostic and screening utilities. In one aspect, the invention provides a method for facilitating hair growth in a tissue containing a hair follicle comprising administering to the tissue during the telogen phase of the hair follicle. In one aspect, they are administered an effective amount of an agent that inhibits Bone Morphogenic Protein (BMP) signaling in the tissue, thereby facilitating hair growth. In one aspect, the method further comprises determining the phase of hair growth prior to administration of the agent and identifying when the hair follicles are in the telogen phase of hair growth.

In another aspect, the invention provides a method for treating alopecia in a subject having tissue containing a hair follicle, comprising administering to the tissue an effective amount of an agent that inhibits Bone Morphogenic Protein (BMP) signaling to the tissue, thereby treating alopecia in the subject. In one aspect, they are administered during the telogen phase of hair growth. In one aspect, the method further comprises determining the phase of hair growth prior to administration of the agent and identifying when the hair follicles are in the telogen phase of hair growth.

In the above methods, the agent that inhibits BMP is one or more selected from the group of BMP antagonists including noggin, chordin, gremlin, sclerostin, follistatin, a small interference RNA (siRNA) or double strand RNA (dsRNA) that inhibits one or more genes selected from the group consisting of BMP1, BMP2, BMP3, BMP4, BMP5, BMP6, BMP7, BMP8a, BMP8b, BMP10 and BMP15, or an antibody or modified antibody that inhibits a BMP antagonist or activates or stabilizes a BMP protein selected from the group consisting of BMP1, BMP2, BMP3, BMP4, BMP5, BMP6, BMP7, BMP8a, BMP8b, BMP10 and BMP15.

In a further aspect, the methods further comprise administering an effective amount of one or more of monoxidal, finasteride, spironolactone or a second agent enhancing hair growth.

In a further aspect, the invention provides a method for inhibiting hair growth in a tissue containing a hair follicle comprising administering to the tissue an effective amount of an agent that augments Bone Morphogenic Protein (BMP) signaling in the tissue, thereby inhibiting hair growth. In a further aspect, the agent is administered during the telogen phase of the hair follicle.

In a yet further aspect, the invention provides a method for treating hirsutism in a subject having tissue containing a hair follicle, comprising administering to the tissue of the hair follicle an effective amount of an agent that augments Bone Morphogenic Protein (BMP) signaling in the tissue, thereby treating hirsutism in the subject. In a further aspect, the agent is administered during the telogen phase.

For these methods, the agent that augments BMP is selected from the group consisting of isolated or recombinant BMP1, BMP2, BMP3, BMP4, BMP5, BMP6, BMP7, BMP8a, BMP8b, BMP10 and BMP15 proteins and combinations thereof, or alternatively one or more selected from an isolated or recombinant polypeptide of BMP1, BMP2, BMP3, BMP4, BMP5, BMP6, BMP7, BMP8a, BMP8b, BMP10 and BMP15 or one or more selected from a polypeptide agonist of BMP1, BMP2, BMP3, BMP4, BMP5, BMP6, BMP7, BMP8a, BMP8b, BMP10 and BMP15. In a further aspect, the agent is an antibody or modified antibody that activates or stabilizes a BMP antagonist or inhibits a BMP protein selected from the group consisting of BMP1, BMP2, BMP3, BMP4, BMP5, BMP6, BMP7, BMP8a, BMP8b, BMP10 and BMP15. Agonists include repulsive guidance molecule (RGMA), DRAGON (RGMB), hemojuvelin, kielin/chordin-like protein (KCP), and Crossveinless 2 (Cv2).

In each of the above methods, the method may further comprise ablating the tissue prior to administration of the agent and/or further comprises administration of penetration enhancer prior to or concomitantly with administration of the agent.

In each of the above methods, administration of the agents may be by any one or more of topical or interdermally, using creams, gels, solutions, sprays, microneedles or ionotophoresis or the like.

Compositions described above, such as small molecules BMP agonists or antagonists, polynucleotides and polypeptides both agonistic and antagonistic, can be administered to the subject in need of. In one aspect, the composition is directly delivered into or onto the skin. In another aspect, the composition is delivered during telogen phase or during competent telogen phase of the hair follicle which can be determined by one skilled in the art and briefly described herein. In another aspect, delivery can be made via microneedles. Microneedles allow penetrating stratum corneum—the outer layer of epidermis, responsible for the most of skin's barrier properties. Since microneedles do not reach into deeper skin layer, they do not cause painful sensations.

BMP proteins have been successfully delivered intracutaneously via single glass microneedles. Delivery of BMP proteins during competent telogen phase rendered treated skin refractory and prevented hair regeneration. For more standardized and simplified intracutaneous delivery hollow microneedle arrays can be used. Microneedle arrays contain hundreds of small individual microneedles evenly spaced apart on a platform. Microneedle array can also be connected to protein reservoir and injection mechanism. Such delivery apparatus can be realized in form of disposable injection syringe. Alternative delivery platform can be based on principle of micro-fluidics. Microneedle/micro-fluidics device will provide slow intradermal delivery of compound at a constant rate over prolonged period of time. Such delivery platform can be realized in form of skin patch that can be attached over treatment area and worn without inconvenience for the patient.

Microneedles are commonly produced as multineedle arrays from silicon, metal, glass via means of micro-etching. Microneedles are designed to be 100 to 1000 mkm in length. When applied to the skin, micro-needle arrays puncture through stratum corneum into deeper layers of epidermis, while not penetrating all the way into the dermis. Thus, they effectively disrupt stratum corneum barrier, and yet at the same time to not reach cutaneous nerve endings or the capillaries, preventing pain, bleeding skin infection.

Micro-needles can be solid or hollow. If solid micro-needles are used, drug is applied to the skin in the form of spray, or gel upon removal of the micro-needle array. Use of hollow needles will allow direct passive drug delivery via produced micro-conduits. The active agent can be dry coated onto the inner surface of the micro-needles. It can also be co-administered as solution, suspension, emulsion or gel. Furthermore, use of hollow micro-needle arrays enable active drug delivery via combination of micro-needle array with microfluidic devices. These methods of stratum corneum disruption allow effective delivery of large molecular weight compounds such as peptides, proteins, and DNA constructs.

Microneedle arrays can be combined with syringe-like injection device to achieve simple protein delivery. Such delivery system can be realized in form of dermal patch, similar to ionophoretic insulin dermal patch.

Expression vectors, such as those expressing BMP ligands or antagonists, or naked cDNA for these genes can be delivered into skin using established intracutaneous gene delivery techniques, such as technique of electorporation or with the help of “gene gun”. In order to express the proteins described herein, delivery of nucleic acid sequences encoding the gene of interest can be delivered by several techniques as described herein. A polynucleotide can be delivered to a cell or tissue using a gene delivery vehicle. Gene delivery vehicles may also include DNA/liposome complexes, micelles and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens. Cell surface antigens characteristic to epidermis or hair follicle specific cell types should be used. Alternatively, antigens characteristic to stem cells should be used to target gene delivery into stem cells (such as hair follicle stem cells). In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

In one aspect, the composition for use in the methods further comprises a penetration enhancer or a carrier suitable for controlled release. Examples of penetration enhancers include, for example, propylene glycol/lauric acid, linalool, alpha terpineaol, carvacrol, limonene, menthone, eugenol, phloretin, polyphenol. The compositions can be formulated for delivery by spraying, topical administration, in a hydrogel or a transdermal patch.

In some embodiments, compositions of the invention can be delivered into the skin by injection with a carrier for long term release and effect. In one aspect, beads are used as a protein reservoir. In another aspect, the composition further comprises a biocompatible and/or dissolvable carrier. Non-limiting examples of biocompatible and/or dissolvable carriers include injectable collagen matrix, dissolvable hydrogel and injectable biocompatible and dissolvable polymers.

In one aspect, the composition of this invention can be used to treat a condition in a mammalian subject in need of. In some embodiments, the condition comprises excessive hair, excessive hair growth, hair loss or insufficient hair growth. In one aspect, the condition is alopecia. In another aspect, condition is hirsutism. In one aspect, the invention provides methods to prevent alopecia. In another aspect, the invention provides methods to prevent hirsutism. In one aspect, the invention provides methods to enhance BMP signaling in the skin. In another aspect, the invention provides methods to inhibit BMP signaling in the skin.

In another aspect, the composition or compositions can be co-administrated, or administered prior to or after administration of a second agent that enhances or inhibit hair growth. In one aspect, the second agent is minoxidil, a treatment for alopecia, commercially available as Rogaine or Regaine. In some embodiments, a combination of slow release excipients having two different rates of release where the composition of the invention is released over the course of a few hours, a day or more, followed by several days of release of the second agent. In another aspect, time release encapsulation comprising the compositions of the invention can be included in shampoo for convenient administration.

One aspect of the invention provides a method to determine if a test agent will likely modulate hair growth in a tissue having a hair follicle, comprising: (a) administering to a first tissue sample an amount of the test agent; (b) administering to a second tissue sample an effective amount of soluble BMP and/or (c) administering to a third tissue sample an effective amount of BMP antagonist, such as noggin; and (d) comparing the growth of hair in the first tissue sample to the growth in the second tissue sample and/or third tissue sample, wherein the test agent will likely modulate hair growth if the growth of hair in the first tissue sample is similar to the second tissue sample and/or third sample. In some embodiments, the method further comprises laser ablating or tape stripping of the tissue prior to administration of the agents. In yet some other aspects, the method further comprises administration of penetration enhancer prior to or concomitantly with administration of the agents.

The invention in one aspect provides a method to determine if a test agent will likely facilitate hair growth in a tissue of an animal, which animal comprises an expression cassette stably integrated into the animal genome, which expression cassette comprises a polynucleotide encoding a BMP protein under control of a skin-specific promoter, and then administering to the tissue an effective amount of the test agent, wherein formation of new hair or an increase of hair growth indicates that the test agent will likely facilitate hair growth. In some embodiments, the BMP protein is selected from the group consisting of BMP1, BMP2, BMP3, BMP4, BMP5, BMP6, BMP7, BMP8a, BMP8b, BMP10 and BMP15.

The invention in another aspect provides a method to determine if a test agent will likely inhibit hair growth in a tissue of an animal, which animal comprises an expression cassette stably integrated into the animal genome, which expression cassette comprises a polynucleotide encoding a BMP antagonist under control of a skin-specific promoter, comprising administering to the tissue an effective amount of the test agent, wherein formation of new hair or an increase of hair growth indicates that the test agent will likely inhibit hair growth. In some embodiments, the BMP antagonist is selected from the group consisting of dorsomorphin, noggin, chordin, gremlin, sclerostin and follistatin. In one particular aspect, the BMP antagonist is noggin. In a further aspect the method is preformed in combination with additional agonist or antagonists as described above. The additional agents can be co-administered or delivered prior to or after the other agent. In a yet further aspect, positive and negative controls are added. In a further aspect, the method is performed during the telogen phase of the hair follicles.

One aspect of the invention provides a method for determining if a subject having a condition is suitable for a treatment targeting BMP signaling, which condition comprises alopecia, which treatment comprises administration of an agent inhibiting BMP signaling, wherein an expression level of BMP mRNA or protein lower than a predetermined value indicates that the subject is suitable for the treatment. In a particular aspect, the BMP gene is BMP2. In another aspect, the BMP gene is BMP4.

Another aspect of the invention provides a method for determining if a subject having a condition is suitable for a treatment targeting BMP signaling, which condition comprises hirsutism, which treatment comprises administration of an agent augmenting BMP signaling, wherein an expression level of BMP mRNA or protein higher than a predetermined value indicates that the subject is suitable for the treatment. In a particular aspect, the BMP gene is BMP2. In another aspect, the BMP gene is BMP4.

In some embodiments, the predetermined value for evaluating BMP protein or mRNA expression level is determined in a subject, by comparing the areas of skin having high or low hair growth. A value that best separates expression values into a high hair growth group and a low hair growth group is the predetermined value. In some other embodiments, the predetermined value for evaluating BMP protein or mRNA expression level is determined in a group of subjects, by comparing the subjects with high hair growth to subjects with low hair growth. A value that best separates expression values into a high hair growth group and a low hair growth group is the predetermined cutoff value. In some embodiments, the subject is human.

mRNA expression values of a BMP gene may be determined with technology well known in the art. Examples of such technologies, without limitation, include real time PCR, in situ hybridization and microarray. In one aspect, the technology is real time PCR. Non-limiting examples of primers and probes to be used in real time PCR for human BMP genes include primer/probe sets commercially available from Applied Biosystems (Foster City, Calif.): BMP1 (Assay ID: Hs00241807_ml), BMP2 (Assay ID: Hs00154192_ml), BMP3 (Assay ID: Hs00609639_ml), BMP4 (Assay ID: Hs00370078_ml), BMP5 (Assay ID: Hs00951007_ml), BMP6 (Assay ID: Hs00233470_ml), BMP7 (Assay ID: Hs00233476_ml), BMP8a (Assay ID: Hs00426893_g1), BMP8b (Assay ID: Hs01629120_s1), BMP10 (Assay ID: Hs00205566_ml) and BMP15 (Assay ID: Hs00193764_ml).

The agents and compositions of the present invention in all aspects as described above can be used in the manufacture of medicaments and for the treatment of humans and other animals by administration in accordance with conventional procedures, such as an active ingredient in pharmaceutical compositions.

The following examples are provide to illustrate select embodiments of the invention as disclosed and claimed herein.

### EXPERIMENTAL EXAMPLES

### Example 1

### Modulation of Hair Growth Via Positive or Negative Regulation of BMP Signaling

### Methods:

### Animals

C75BL/6J, Crl:CD1(ICR), C3H/HeJ and SCID mice were used in this study. Msx2 null (C.Cg-Msx2^{tm1Rilm}/Mmcd), Krt14-Nog (B6,CBA-Tg(Krt14-Nog)), Bmp4-lacZ (129S-Bmp4^{lacZneo}), Nog-lacZ (129S-Nog^{tm1Amc}/J) and TOPGAL (STOCK Tg(Fos-lacZ)34Efu/J) transgenic mice were also used.

### Hair-Cycle Observation

Progression of hair growth patterns was monitored in mice for various intervals of time, up to 1 year. Hair clipping was selected over plucking or shaving to avoid wounding that can potentially interfere with normal hair growth (Plikus and Chuong (2008) J. Invest. Dermatol. 128(5):1071:80, Chase (1954) Physiol. Rev. 34:113-26).

### Animal Procedures

All procedures were performed on anaesthetized animals with protocols approved by USC vivaria. For skin transplantation, surgical procedures were performed when both donor and recipient skins were in early telogen. This was done to ensure that wounded skin is healed by the beginning of the next anagen phase and that the affect of wound healing on the hair cycle is minimal. SCID mice were used as recipients.

### Histology and Detection of Molecular Expressions

Tissues were collected, fixed and processed for histology as described (Plikus and Chuong (2008) J. Invest. Dermatol. 128(5):1071:80, Plikus et al. (2004) Am. J. Pathol. 164:1099-114).

### Choosing Early Versus Late Telogen Skin

To choose early versus late telogen skin in living mice, the following protocol was used.

First, an area on the adult mouse skin where hairs appeared to be growing was chosen. The use of pigmented mice made it easier to distinguish these phases. Hairs were clipped (not plucked) near the skin surface. Anagen-phase skin contains pigment in the proximal hair follicles. This determination can be aided by observing the skin under a dissection microscope, especially when the skin is wet with saline solution to make it appear transparent. These mice were monitored daily, and the day on which skin pigmentation ceased was recorded. This coincides with the anagen/catagen junction. A wait of an additional 5 days to ensure that skins are in early telogen provides early telogen skin to work with. Alternatively, 40 days or longer (well over 4 weeks) after the anagen/catagen junction for late telogen skin to develop provides late telogen skin to work with.

### Scoring the Plucking Experiments

Hairs were plucked from the early or late telogen region. After plucking, each plucked spot was monitored daily under a dissection microscope. New anagen skin on living mice was detected without having to biopsy or kill the mice for histological specimens. Changes were checked in pigmentation since the start of melanogenesis in anagen III. Pigmented hairs can be spotted under a dissection microscope before the new hair fibres reach the skin surface. Thus, the appearance of anagen III hair follicles (when we spotted black hairs under the skin surface) was recorded non-invasively. Approximately, this corresponds to the second day of new anagen. It takes another day for the new hair fibre to reach the skin surface. Thus, day-3 anagen follicles was also recorded non-invasively when the new hair filaments reach above the skin surface.

Because the changes in skin pigmentation are not easily visible, the appearance of new hair filaments above the skin surface was used as the criteria for scoring hair-plucking experiments. Therefore, it takes approximately 9 days to observe the appearance of day-3 anagen follicles. The extra time includes the period required for the follicle to heal and to get ready to enter anagen.

### Protein Administration Experiment

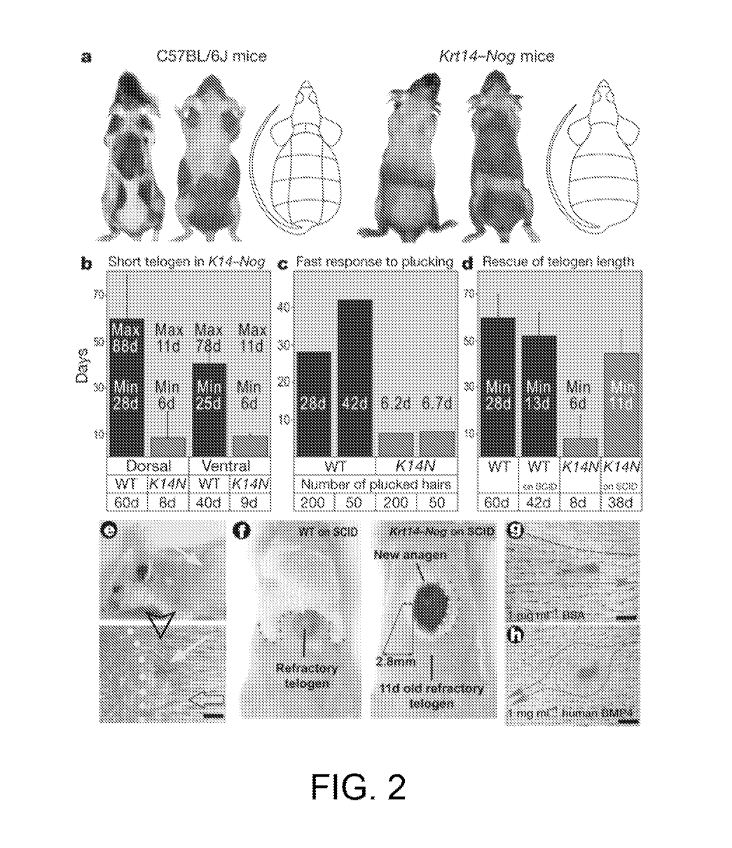

Intracutaneous administration of exogenous protein was performed as follows. Affinity chromatography Affi-gel blue gel beads were obtained from Biorad. Beads were washed in 1×PBS, followed by drying. The beads were then re-suspended in 5 μl protein solution, either control (BSA 1 mg ml⁻¹) or experimental (human BMP4 1 mg per ml), at 4° C. for 30 min. Recombinant human BMP4 protein was obtained from R&D Systems. Reconstitution of the protein was performed in 4 mM HCl in 0.2% BSA as per the manufacturer's guidelines. Approximately 100 beads were introduced to the competent telogen skin of adult mice by means of a single puncture wound to the skin made by a 30 g syringe (insulin syringe). To replenish proteins, subsequent doses of 1.5 μl protein solution were microinjected to the site of the bead implantation every 24 h by means of a glass micro-needle until the tissue was harvested. After the anagen-spreading wave was noted to pass beyond the bead implantation sites (1 week in the case of FIGS. 2g, h), the skin was collected and inverted for photography. This allows the study of the anagen-wave-spreading dynamics around the control and human BMP4 beads.

### Results:

Mammalian skin contains thousands of hair follicles, each undergoing continuous regenerative cycling. A hair follicle cycles through anagen (growth), catagen (involution) and telogen (resting) phases, and then re-enters the anagen phase. At the base of this cycle is the ability of hair follicle stem cells to briefly exit their quiescent status to generate transient amplifying progeny, but maintain a cluster of stem cells. It is generally believed that a niche microenvironment is important in the control of stem cell homeostasis in various systems (Moore & Lemischka (2006) Science 311:1880-5). It has been suggested that skin regions in telogen can be in either of the two functional phases: competent telogen, which allows the anagen-re-entry wave to propagate, and refractory telogen, which arrests the wave. In mice, there is a minimal 28-day-long telogen phase which is referred to as early telogen. After this phase, telogen can either end right away (0 days) or persist for any number of days up to about 60 days (late telogen) which contributes to the apparently highly variable telogen length. Understanding the molecular mechanisms underlying these dynamic and complex hair growth patterns would provide valuable information for the treatment of various hair growth conditions such as alopecia and hirsutism.

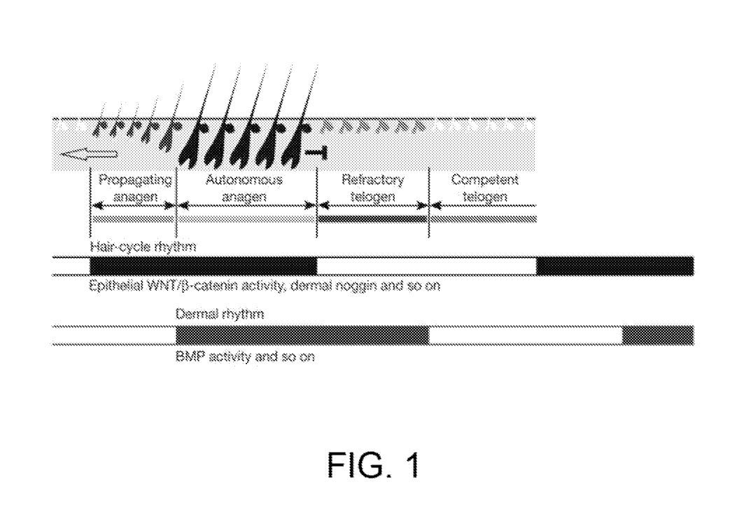

It has been recently discovered by the inventors that cyclic activity of hair follicles is largely regulated by signaling molecules normally expressed in the dermal macro-environment (Plikus et al. (2008) Nature 451(7176):340-4). Expression of BMP, at both mRNA and protein levels, was negatively correlated with hair growth. In mouse dermis with complex hair cycle domain patterns, an unexpected periodic expression of BMP2 and BMP4 proteins was observed and was shown to regulate the cyclic process (Plikus et al. (2008) Nature 451(7176):340-4 and Plikus and Chuong (2008) J. Invest. Dermatol. 128(5):1071-80) (FIG. 1). The content of these articles are herein incorporated by reference in their entirety.

If BMPs have a causative role in conferring refractory status, then is it possible to reduce the period of refractory telogen by down-regulating BMP signaling? To answer this question, the Nog gene was overexpressed in mice under the keratin 14 promoter in Krt14-Nog mice (named K14-Noggin in Plikus et al. (2004) Am. J. Pathol. 164:1099-114). The minimal telogen length was reduced to 6 days, and the maximal length was reduced to 11 days (FIG. 2). As a result, these mice displayed continuous propagation of hair regenerative waves and have highly simplified hair-cycle-domain patterns (FIG. 2a). The response of Krt14-Nog hair follicles to hair plucking was also tested. Hair plucking generally stimulates hair regeneration (Plikus et al. (2008) Nature 451(7176):340-4). The differences in response we observed in wild-type mice in early versus late telogen were eliminated in Krt14-Nog mice. In all cases, plucked Krt14-Nog hair follicles required only approximately 6 days to re-enter anagen (FIG. 2c). The importance of BMP activity in suppressing stem cell activity has also been shown by tissue-specific deletion of BMP receptors (Kobielak et al. (2007) Proc. Natl. Acad. Sci. 104:10063-68, Zhang et al. (2006) Stem Cells 24:2826-39).

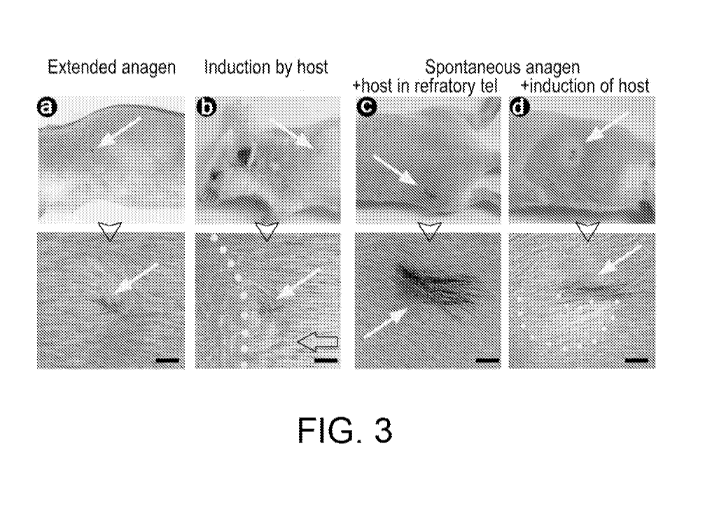

The currently held concept of the stem cell microenvironment implies only autonomous regulation: thus, the activation of stem cells depends only on signaling inputs from components intrinsic to the organ (here, the hair follicle itself (Fuchs (2004) et al. Cell 116:769-78). To test directly whether the activation of stem cells is also subjected to non-autonomous regulation, skin grafts from pigmented Krt14-Nog mice were transplanted onto albino severe combined immunodeficient (SCID) mice. If the control of stem cell activation is intrinsic to the follicles, hair cycling behaviour should remain the same for both donor and host. Instead, donor-host interactions were observed, reflecting a non-autonomous relationship, with the outcome dependent on the size of the transplanted skin graft. When a small graft of Krt14-Nog skin (˜1 mm) was transplanted, the donor skin remained in telogen for longer and could respond to an anagen-activating wave originating from the host (FIGS. 2e and 3). Thus, partial functional rescue of Krt14-Nog phenotypes was achieved. In contrast, when a large skin graft (>10 mm) was transplanted, the graft exhibited a greater degree of autonomous control within itself. Host telogen hair follicles surrounding the graft re-entered anagen (visible as a rim of white hairs) when pigmented donor hairs entered anagen (FIG. 2f) after only 11 days in telogen (versus 28 days), thus providing evidence of a donor effect on the host.

Classical experiments using skin graft transplantation to ask whether hair growth patterns are controlled intrinsically or systemically have produced variable results (Ebling et al. (1961) J. Embryol. Exp. Morphol. 9:285-93). Autologous skin transplantation experiments showed that hair growth patterns are initially intrinsic to the donor but gradually become entrained to the host rhythm after several hair cycles. Consequently, the discrepancy amongst classical experiments may be due to the size of the graft and the time they chose for readout. At the molecular level, these results demonstrate involvement of the BMP pathway in the non-autonomous interactions among follicle populations. It remains to be investigated whether the process depends on the direct diffusion of BMPs or their antagonists, or whether it is indirectly mediated by other mechanisms (Oro and Higgins (2003) Dev. Biol. 255:238-248).

Finally, it was tested whether a direct local delivery of BMP protein can convert competent telogen status to refractory in normal mice. Human-BMP4-soaked beads were implanted into competent telogen skin ahead of an anagen-spreading wave (see Methods in Botchkarev et al. (2001) FASEB J. 15:2205-14). Twelve days later, human BMP4, but not control BSA, prevented the propagation of the wave around the beads (FIGS. 2g, h and 4). Thus, the level of BMP activity can indeed explain the functional status (refractory versus competent) of a skin region.

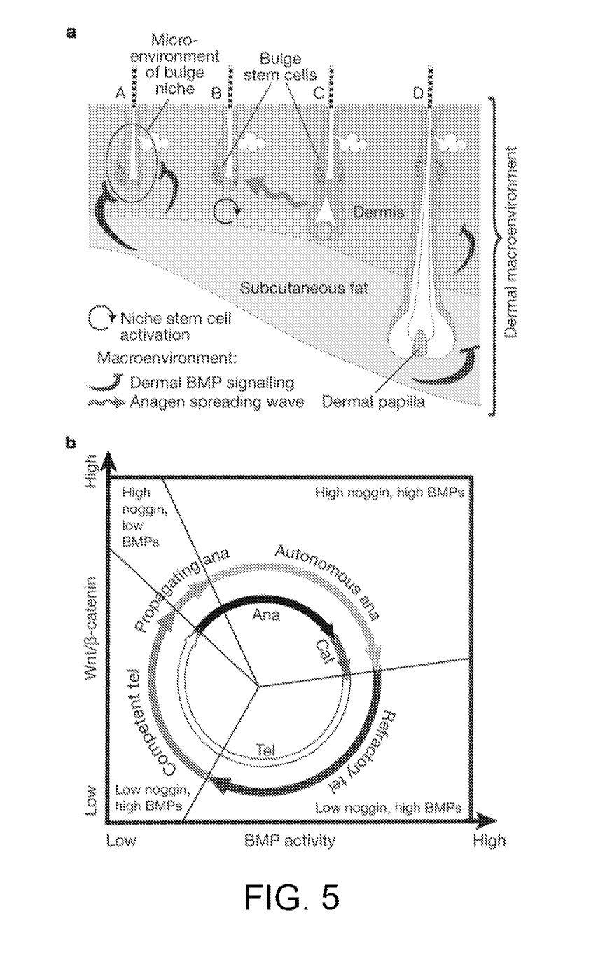

Results here add new dimensions to the understanding of skin biology. First, these findings demonstrate that, in addition to short distance microenvironmental control (Botchkarev et al. (2001) FASEB J. 15:2205-14, Blanpain et al. (2004) Cell 118:635-48), the activation of stem cells within large groups of hair follicles is subject to long distance macroenvironmental control from the surrounding dermis (FIG. 5). This concept is readily applicable to other organs. For example, whereas Bmp4 is constantly expressed in the mesenchyme of intestinal microvilli, bursts of Nog expression in the villi stem cell niche may act transiently to lower BMP signalling, thus allowing stem cells to proliferate for epithelial renewal (He et al. (2004) Nature Genet. 36:1117-21). Second, extrafollicular periodically expressed Bmp2 and Bmp4 seem to fulfill the criteria of the elegant but elusive chalone proposed to explain patterned hair growth (Ebling and Johnson (1961) Embryol. Exp. Morphol. 9:285-93, Chase (1954) Physiol. Rev. 34:113-26, Botchkarev et al. (2001) FASEB J. 15:2205-14), thus solving a 50-year-old puzzle. Third, the dynamic expression of Bmp2 in dermal adipocytes suggests a link between two skin organ systems. Because subcutaneous fat, like hairs, has a thermo-regulatory function and leptin is present in the dermal papilla of hair follicles (Iguchi et al. (2001) J. Invest. Dermatol. 117:1349-56), periodically expressed Bmp2 may coordinate the function of these two organs in response to the external environment and may have implications for the evolution of integuments (Wu et al. (2004) Int. J. Dev. Biol. 48:249-70). Fourth, the asynchronous cyclic expression of BMPs and beta-catenin in the dermis and hair follicle provide a platform for mutual modulations of these ‘clocks’ in the skin. They also imply that stem cell regeneration is subject to the control of biological rhythms.

These results show that Bone Morphogenic Protein (BMP) ligands: BMP2 and BMP4 when expressed in dermal macro-environment during telogen (resting phase of hair cycle) strongly suppress ability of resting hair follicles to be reactivated to grow again.

It also shows that natural hair growth can be enhanced or suppressed via alteration in dermal macro-environment, which is defined as the environment outside the hair follicles themselves, and includes dermis and subcutaneous tissue. These findings demonstrate that cyclic activity of hair follicles is largely controlled by the inhibiting and activating signaling molecules normally expressed in the dermal macro-environment.

Levels of BMP activity in skin can be altered artificially either via over-expression or direct delivery of BMP proteins or genes as well as various known BMP antagonists, such as noggin (UniProt: Q13253) which can be produced using methods described in, e.g. McMahon et al. (1998) Genes & Development 12:1438-52, Chordin, Gremlin, Sclerostin and Follistatin. Lowering of BMP signaling produces dermal environment that is very permissive and even inductive for hair growth. Increasing BMP signaling results in dermal macroenvironment that strongly suppresses hair growth. This can be utilized to modulate hair growth which can become especially useful in treatment of alopecia and hirsutism.

The dermal macroenvironment can modulate activity of stem cells in thousands of hair follicles at the same time. This is accomplished via expression of soluble BMP ligands. When highly expressed during resting phase, these macroenvironment-derived BMP ligands strongly inhibit regeneration of all follicles included in the BMP expressing skin domain. When BMP ligands are largely absent, macroenvironment becomes permissive and resting follicles can regenerate.

Current technologies aim to enhance or suppress hair growth work by targeting hair follicles directly. The new mechanism disclosed in this invention is based on regulation of hair growth via modification in inter-follicular dermal macro-environment. Thus the approach in this invention is more physiological. Also, because changes in macro-environmental either local or systemic can affect hair cycling in thousands follicles at the same time, this creates possibility to manipulate regeneration within large areas of skin simultaneously.

Hair regeneration can be either facilitated or inhibited through modulation of dermal macro-environment. In turn, competent and refractory status of dermal macro-environment can be modulated via intracutaneous gene overexpression, protein or pharmaceutical compound delivery.

Small molecular weight compounds, such as mimetics of BMP antagonists can be delivered via simple application onto the surface of the skin. In this case, BMP inhibitory action of such compound(s) will counteract BMPs secreted from dermal macro-environment. At the level of hair follicles, this can reduce strength of BMP signaling and render hair follicles competent to regeneration. Hair follicles will enter growth phase despite otherwise refractory macro-environment. This will imitate situation observed in KRT14-noggin mouse, where excess of transgenic noggin is overexpressed in epidermis and it counteracts inhibitory activity of BMPs in dermal macro-environment. Hairs in KRT14-noggin mice regenerate very fast skipping refractory telogen phase.

As discussed above, a potent small molecular weight Bmp signaling antagonists, Dorsomorphin (aka compound C), has been reported (Hao et al. (2-8) PLoS ONE 3(8):e2904, Yu et al. (2008) Nat. Chem. Biol. 4(1):33-41).

Small molecule agonist and antagonists for other signaling pathways exist. Interaction of these small molecules with pathways including, but not limited to, WNT, SHH and FGF will also have direct or indirect impact on BMP signaling thus serve as effective modulator of hair growth via methods disclosed in this invention.

Proteins, both agonistic and antagonistic, can be directly delivered into skin. A preferred delivery method is via microneedles. Microneedles allow penetrating stratum corneum—the outer layer of epidermis, responsible for the most of skin's barrier properties. Since microneedles do not reach into deeper skin layer, they do not cause painful sensations.

BMP4 protein has been successfully delivered intracutaneously via single glass microneedle. Delivery of BMP4 protein during competent telogen phase rendered treated skin refractory and prevented hair regeneration. For more standardized and simplified intracutaneous delivery hollow microneedle arrays can be used. Microneedle arrays contain hundreds of small individual microneedles evenly spaced apart on a platform. Microneedle array can also be connected to protein reservoir and injection mechanism. Such delivery apparatus can be realized in form of disposable injection syringe. Alternative delivery platform can be based on principle of micro-fluidics. Microneedle/micro-fluidics device will provide slow intradermal delivery of compound at a constant rate over prolonged period of time. Such delivery platform can be realized in form of skin patch that can be attached over treatment area and worn without inconvenience for the patient.

Microneedle arrays can be combined with syringe-like injection device to achieve simple protein delivery. Such delivery system can be realized in form of dermal patch, similar to ionophoretic insulin dermal patch.

Expression vectors, such as those expressing BMP ligands or antagonists, or naked cDNA for these genes can be delivered into skin using established intracutaneous gene delivery techniques, such as technique of electorporation or with the help of “gene gun”.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for facilitating hair growth in a tissue containing a hair follicle comprising administering to the tissue an effective amount of an agent that inhibits Bone Morphogenic Protein (BMP) signaling in the tissue, thereby facilitating hair growth.

2. A method for treating alopecia in a subject having tissue containing a hair follicle, comprising administering to the tissue an effective amount of an agent that inhibits Bone Morphogenic Protein (BMP) signaling to the tissue, thereby treating alopecia in the subject.

3. A method for inhibiting hair growth in a tissue containing a hair follicle comprising administering to the tissue an effective amount of an agent that augments Bone Morphogenic Protein (BMP) signaling in the tissue, thereby inhibiting hair growth.

4. A method for treating hirsutism in a subject having tissue containing a hair follicle, comprising administering to the tissue an effective amount of an agent that augments Bone Morphogenic Protein (BMP) signaling in the tissue, thereby treating hirsutism in the subject.

5. The method of claim 1, wherein the agent is one or more dorsomorphin, noggin, chordin, gremlin, sclerostin or follistatin.

6. The method of claim 1, wherein the agent is a small interference RNA (siRNA) or double strand RNA (dsRNA) that inhibits one or more genes of BMP1, BMP2, BMP3, BMP4, BMP5, BMP6, BMP7, BMP8a, BMP8b, BMP10 or BMP15.

7. The method of claim 1, wherein the agent is an antibody or modified antibody that inhibits a BMP antagonist or activates or stabilizes a BMP protein is one or more of BMP1, BMP2, BMP3, BMP4, BMP5, BMP6, BMP7, BMP8a, BMP8b, BMP10 or BMP15.

8. The method of claim 1, further comprising administering an effective amount of monoxidal or a second agent enhancing hair growth.

9. The method of any one of claim 3, wherein the agent is one or more of an isolated or recombinant BMP1, BMP2, BMP3, BMP4, BMP5, BMP6, BMP7, BMP8a, BMP8b, BMP10 or BMP15.

10. The method of claim 3, wherein the agent is one or more of an isolated or recombinant polypeptide of BMP1, BMP2, BMP3, BMP4, BMP5, BMP6, BMP7, BMP8a, BMP8b, BMP10 or BMP15.

11. The method of claim 3, wherein the agent is one or more of a BMP1, BMP2, BMP3, BMP4, BMP5, BMP6, BMP7, BMP8a, BMP8b, BMP10 or BMP15.

12. The method of claim 3, wherein the agent is an antibody or modified antibody that activates or stabilizes a BMP antagonist or inhibits a BMP protein selected from BMP1, BMP2, BMP3, BMP4, BMP5, BMP6, BMP7, BMP8a, BMP8b, BMP10 or BMP15.

13. The method of claim 1, further comprising ablating the tissue prior to administration of the agent.

14. The method of claim 1, wherein the agent is administered topically.

15. The method of claim 14, further comprising administration of penetration enhancer prior to or concomitantly with administration of the agent.

16. The method of claim 1, wherein the subject is a human patient.

17. A composition for inhibiting hair growth comprising an effective amount of an agent that augments BMP signaling and a pharmaceutically acceptable carrier.

18. A composition for inhibiting hair growth comprising an effective amount of an agent that inhibits BMP signaling and a pharmaceutically acceptable carrier.

19. The composition of claim 15, wherein the carrier is suitable for topical administration of the agent.

20. The composition of claim 19, further comprising a penetration or permeation enhancer.

21. (canceled)

22. (canceled)

23. A method to determine if a test agent will likely modulate hair growth in a tissue having a hair follicle, comprising:
(a) administering to a first tissue sample an amount of the test agent;
(b) administering to a second tissue sample an effective amount of soluble BMP and/or
(c) administering to a third tissue sample an effective amount of BMP antagonist; and
(d) comparing the growth of hair in the first tissue sample to the growth in the second tissue sample and/or third tissue sample, wherein the test agent will likely modulate hair growth if the growth of hair in the first tissue sample is similar to the second tissue sample and/or third sample.

24. The method of claim 23, further comprising ablating the tissue prior to administration of the agents.

25. The method of claim 24, further comprising administration of penetration enhancer prior to or concomitantly with administration of the agents.

26. A method for determining if a test agent will likely facilitate hair growth in a tissue of an animal, which animal comprises an expression cassette stably integrated into the animal genome, which expression cassette comprises a polynucleotide encoding a BMP protein under control of a skin-specific promoter, comprising administering to the tissue an effective amount of the test agent, wherein formation of new hair or an increase of hair growth indicates that the test agent will likely facilitate hair growth.

27. The method of claim 26, wherein the BMP protein is one or more of BMP1, BMP2, BMP3, BMP4, BMP5, BMP6, BMP7, BMP8a, BMP8b, BMP10 or BMP15.

28. A method for determining if a test agent will likely inhibit hair growth in a tissue of an animal, which animal comprises an expression cassette stably integrated into the animal genome, which expression cassette comprises a polynucleotide encoding a BMP antagonist under control of a skin-specific promoter, comprising administering to the tissue an effective amount of the test agent, wherein formation of new hair or an increase of hair growth indicates that the test agent will likely inhibit hair growth.

29. The method of claim 28, wherein BMP antagonist is one or more of noggin, chordin, gremlin, sclerostin and follistatin.

30. A method for determining if a subject having a condition is suitable for a treatment targeting BMP signaling, which condition comprises alopecia, which treatment comprises administration of an agent inhibiting BMP signaling, wherein an expression level of BMP mRNA or protein lower than a predetermined value indicates that the subject is suitable for the treatment.

31. A method for determining if a subject having a condition is suitable for a treatment targeting BMP signaling, which condition comprises hirsutism, which treatment comprises administration of an agent augmenting BMP signaling, wherein an expression level of BMP mRNA or protein higher than a predetermined value indicates that the subject is suitable for the treatment.

32. The method of claim 30, wherein the BMP is BMP2 or BMP4.

33-36. (canceled)
